(19) United States
(12) Patent Application Publication
(10) Pub. No.: US 2014/0248294 A1
(43) Pub. Date: Sep. 4, 2014

(54) WNT4 AND MED12 FOR USE IN THE DIAGNOSIS AND TREATMENT OF TUMOR DISEASES

(71) Applicant: University of Bremen, Bremen (DE)

(72) Inventors: Jörn Bullerdiek, Bremen (DE); Dominique N. Markowski, Bremen (DE)

(73) Assignee: UNIVERSITY OF BREMEN, Bremen (DE)

(21) Appl. No.: 14/349,830

(22) PCT Filed: Oct. 5, 2012
(86) PCT No.: PCT/EP2012/069737

§ 371 Date: Apr. 4, 2014

(30) Foreign Application Priority Data
Oct. 5, 2011 (EP) 111839635

(51) Int. Cl. C07K 16/18; G01N 33/574; C12Q 1/68; C12N 15/113

(52) U.S. Cl. CPC C07K 16/18; C12N 15/113; G01N 33/57496; C12Q 1/6886
USPC 424/174.1; 514/44 A; 514/44 R; 435/7.1; 530/389.7; 536/24.31; 536/24.33; 435/6.11; 435/6.12

(57) ABSTRACT

Provided are novel methods and compositions for the diagnosis, prognosis and treatment of gynecological tumors, in particular uterine leiomyoma (UL). Furthermore, novel methods and compositions for the treatment of diseases characterized by an aberrant growth of mesenchymal stem cells and their descendants and for the treatment of pituitary and prostate tumors are described.

### FIELD OF THE INVENTION

The present invention generally relates to the detection of characteristic mutations in genes associated with aberrant cell growth and with the provision of novel means in the treatment of corresponding diseases. In particular, the invention relates to methods for determination of the response potential of specific tumors to selected kinds of treatment, for the estimation of the growth potential of the tumors characterized by defined gene mutations and for the differential diagnosis of tumors.

### BACKGROUND OF THE INVENTION

Uterine leiomyomas (syn.: fibroids) are among the most frequent clinically relevant human tumors leading, e.g., to abdominal pain, bleeding, and infertility. Their prevalence clearly differs depending on ethnicity but in most countries exceeds 50% of all women in their reproductive ages [1,2]. The monoclonal origin of fibroids [3-6] suggests mutations of myometrial target cells as the cause of the disease. Clonal chromosomal aberrations are found in roughly 20% of the fibroids. Of these, recurrent chromosomal translocations involving chromosomal regions 12q14˜15 or 6p21, respectively, that account for the majority of cytogenetic deviations lead to transcriptional upregulation of the human high mobility group AT-hook (HMGA) genes [7-9] resulting in an activation of the p14^{Arf}-p53 network [10].

Nevertheless, the majority of the fibroids remain without cytogenetically visible changes of the genome. Although only a minority of the leiomyomas becomes symptomatic, the presence of symptomatic leiomyomas is still the leading cause for hysterectomy worldwide. Despite their high prevalence the treatment options besides surgical removal by hysterectomy or tumor enucleation are still limited. Treatment by gonadotropin-releasing hormone (GnRH) agonists as well as antagonists can induce shrinkage of fibroids but re-growth of the tumors usually occurs after termination of the therapy [3, 4]. Thus, intervention at the hormonal level is as a rule only recommended to reduce tumor size pre-operatively [5]. Another alternative represents embolization of the fibroids but the recurrence of myoma-related symptoms is not a rare finding after that treatment as well [6]. Thus, therapies aimed at permanent shrinkage of the fibroids still remain a challenge. Furthermore, diagnostic means are required allowing identification of the mutational origin in the prevalent cases which do not show chromosomal aberrations. In this respect the clarification of the affected gene and/or signalling pathways and accordingly an appropriate classification of the tumors is required for diagnostic means, such as a better prediction of the development of a given tumor and possible base for differential therapy allowing a more specific and effective treatment of the tumor.

The above-mentioned problems are solved by the embodiments characterized in the claims and described further below.

### SUMMARY OF THE INVENTION

The present invention is generally concerned with the detection of characteristic mutations of the mediator sub-complex 12 gene (MED12) for use in the diagnosis of diseases associated with aberrant cell growth and with the provision of novel means in the treatment of said diseases by disclosing the changes of specific cellular characteristics observed in MED12 mutated cells. In particular, tissue isolated from gynaecological tumors, such as fibroids as well as endometrial polyps has been isolated and investigated in respect of chromosomal rearrangements and specific mutations in the MED12 locus and the effects which the occurrence of such genetic aberrations could have on the expression of factors involved in cellular growth, proliferation and differentiation such as the Wnt4 gene.

In this context, experimental results obtained in accordance with the present invention indicate an increased Wnt4 expression in tissue samples isolated from some gynaecological tumors. It appears prudent therefore, to take measures which could ensure a reduction of the Wnt4 expression or even its down-regulation to wild type level, i.e. to levels comparable to these in corresponding non-tumorous, healthy tissue as a therapeutic mean in treatment of such tumors. Thus, the present invention generally relates to Wnt4 inhibitors for use in the treatment of diseases associated with aberrant cell growth such as a benign or malignant gynaecological tumor. The Wnt4 inhibitors may pertain to different classes of molecules, e.g., small molecules, antibodies, antigen-binding fragments of antibodies, aptamers, spiegelmers, siRNA and miRNA and may be used in the treatment of several different tumors such as uterine leiomyoma (UL), endometrial polyps, endometriosis, adenomyosis, leiomyosarcomas of the uterus, aggressive angiomyxomas, endometrial carcinomas and Müllerian mixed tumors.

Furthermore, the present invention provides different methods which are based on the detection of specific mutations affecting the MED12 gene and kits for use in these methods. By detecting and determining one or more MED12 mutations affecting the sequence CAAGGT which will be described in detail further below, methods are disclosed allowing determination of responsiveness of the tested tumor tissue to treatment with Wnt4 inhibitors, wherein in addition or alternatively the Wnt4 expression is determined in the same sample. Furthermore, a method based on the detection and determination of one or more MED12 mutations is provided, which allows the estimation of the growth potential of the tumor tested. Likewise, pituitary tumors, prostate tumors or a prostate hyperplasia may be diagnosed by use of methods of the present invention determining the presence or absence of MED12 mutations as defined hereinbefore and hereinafter in tissue samples isolated from patients.

### BRIEF DESCRIPTION OF THE DRAWINGS

FIG. 1: Chromatograms of the DNA sequences illustrating the different types of MED12 mutations, codon 43 and 44, as detected in 80 uterine fibroids analyzed.
The reference number of the respective tumor (cf. Tab. 1) is shown on the left of each chromatogram and the heterozygous mutation is indicated on the right. The wild type (w.t.) sequence of the fragment shown is given in bold letters above the chromatograms. (A): Results of two independent DNA analyses as well as of cDNA analysis in a case displaying two MED12 mutations. (B): Examples of the different types of MED12 mutations affecting nucleotides c.128, c.130, and c.131. The percentages in grey boxes refer to the frequencies of the corresponding type of mutation among the MED12 mutations observed. Positions of the respective mutations are indicated by arrows.

FIG. 2: The size of uterine fibroids with different types of mutations considerably varies.
(A): Distribution of fibroids with 12q14˜15 rearrangement (black columns) and those with an apparently normal karyotype along with MED12 mutations (white columns) according to three size groups. (B): Distribution of fibroids with an apparently normal karyotype and base transitions of either MED12 c.130 G>A or c.131 G>A (black columns) and those with an apparently normal karyotype along with other MED12 mutations of the “fibroid type” (white columns) according to three size groups.

FIG. 3: Fibroids with MED12 mutation and normal karyotype (white columns) expressed significantly higher amounts of Wnt4 mRNA than those with 12q14˜15 rearrangements (black columns) and normal myometrium (grey columns)
Ordinate: relative expression of Wnt4 mRNA determined by qRT-PCR. For tumor case reference numbers see Tab. 1.

FIG. 4: Differential diagnosis of uterine smooth muscle tumors by using MED12 sequencing, quantification of HMGA2 and MED12 gene expression, and fluorescence in situ hybridization (FISH). Further investigations can be performed as, e.g., FISH for detection of HMGA1 rearrangements in cases where the proposed algorithm does not lead to informative results.

FIG. 5: Important genetic subtypes of human uterine leiomyomas can be found in their canine counterparts as well. (A): Alignment of a part of the human and canine MED12 gene harbouring leiomyoma-like mutations and occurrence of heterozygous MED12 mutations (filled arrows) as revealed by DNA-sequencing of canine vaginal leiomyomas from two dogs (H1,H8). Open arrow indicates a non-conserved nucleic acid in the canine MED12 gene sequence. (B): Gene expression analysis (real-time RT-PCR) reveals two groups of canine leiomyomas characterized by high and low expression of HMGA2 mRNA. Ordinate: relative expression of canine HMGA2 mRNA.

### DETAILED DESCRIPTION OF THE INVENTION

### Definitions

It is to be noted that the term “a” or “an” entity refers to one or more of that entity; for example, “an polypeptide,” is understood to represent one or more polypeptides. As such, the terms “a” (or “an”), “one or more,” and “at least one” can be used interchangeably herein.

### I. Polypeptides

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, dipeptides, tripeptides, oligopeptides, “peptide,” “protein,” “amino acid chain,” or any other term used to refer to a chain or chains of two or more amino acids, are included within the definition of “polypeptide,” and the term “polypeptide” may be used instead of, or interchangeably with any of these terms.

The term “polypeptide” is also intended to refer to the products of post-expression modifications of the polypeptide, including without limitation glycosylation, acetylation, phosphorylation, amidation and derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid residue, e.g., a serine residue or an asparagine residue.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for purposed of the invention, as are native or recombinant polypeptides which have been separated, fractionated, or partially or substantially purified by any suitable technique.

### II. Polynucleotides

The term “polynucleotide” is used interchangeably with the term “nucleic acid molecule”, the use of either of them is intended to encompass a singular nucleic acid as well as plural nucleic acids, and refers to an isolated nucleic acid molecule or construct, e.g., messenger RNA (mRNA) or plasmid DNA (pDNA). A polynucleotide may comprise a conventional phosphodiester bond or a non-conventional bond (e.g., an amide bond, such as found in peptide nucleic acids (PNA)). The term “nucleic acid” refers to any one or more nucleic acid segments, e.g., DNA or RNA fragments, present in a polynucleotide. By “isolated” nucleic acid or polynucleotide is intended a nucleic acid molecule, DNA or RNA, which has been removed from its native environment. For example, a recombinant polynucleotide encoding an antibody contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include in vivo or in vitro RNA transcripts of polynucleotides of the present invention. Isolated polynucleotides or nucleic acids according to the present invention further include such molecules produced synthetically. In addition, polynucleotide or a nucleic acid may be or may include a regulatory element such as a promoter, ribosome binding site, or a transcription terminator.

As used herein, a “coding region” is a portion of nucleic acid which consists of codons translated into amino acids. Although a “stop codon” (TAG, TGA, or TAA) is not translated into an amino acid, it may be considered to be part of a coding region, but any flanking sequences, for example promoters, ribosome binding sites, transcriptional terminators, introns, and the like, are not part of a coding region. Two or more coding regions of the present invention can be present in a single polynucleotide construct, e.g., on a single vector, or in separate polynucleotide constructs, e.g., on separate (different) vectors. Furthermore, any vector may contain a single coding region, or may comprise two or more coding regions, e.g., a single vector may separately encode an immunoglobulin heavy chain variable region and an immunoglobulin light chain variable region. In addition, a vector, polynucleotide, or nucleic acid of the invention may encode heterologous coding regions, either fused or unfused to a nucleic acid encoding a binding molecule, an antibody, or fragment, variant, or derivative thereof. Heterologous coding regions include without limitation specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally may include a promoter and/or other transcription or translation control elements operable associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operable associated” or “operable linked” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operable associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operable associated with the polynucleotide to direct cell-specific transcription. Suitable promoters and other transcription control regions are disclosed herein.

### III. MED12 Mutations and Chromosomal Aberrations

Despite of numerous scientific studies, the majority of the fibroids remains without cytogenetically visible changes of the genome. Quite recently, Mäkinen and colleagues [11] have presented convincing evidence that characteristic mutations of the mediator subcomplex 12 gene (MED12) with a predominance of single base substitutions affecting codon 44 characterize a large subgroup of fibroids. Being part of the preinitiation complex, Mediator forms a large approximately 1.2 MDa aggregate of different subunits involved in the interaction between RNA polymerase II and transcription factors thus performing both general as well as gene-specific roles to activate or repress gene transcription (for review see [12]). Med12 is part of one of the subunits participating in the formation of Mediator and, more specifically, its CDK8 submodule. Alterations of MED12 that has been assigned to Xq13.1 are known to cause the Opitz-Kaveggia and Lujan-Fryns syndrome [13,14] both associated with X-linked mental retardation. The mutations of MED12 now found in leiomyomas are restricted to a different part of the gene. In the study by Mäkinen 159 of 225 lesions (70%) from a total of 80 patients displayed MED12 mutations with a clear predominance of single base substitutions in codon 44. In all tumors analyzed, the mutations were heterozygous and no fibroid showed more than one mutation. cDNA sequencing typically revealed a highly predominant expression of the mutant allele of the X in the tumors. The data represent an important step in understanding the pathogenesis of these highly frequent tumors but at the same time raise a couple of new questions.

It is well documented that fibroids can be subdivided based on the existence of clonal chromosomal aberrations as, e.g., deletions of the long arm of chromosome 7, trisomy 12, or chromosomal rearrangements targeting either of the two human HMGA gene loci (see [15] for a review). From the data presented by Mäkinen et al. it is not clear whether the MED12 mutations coincide with the existence of these karyotypic aberrations or whether they represent independent groups. Mechanistically, Med12 akin to Hmga2 has the ability to influence transcription in a more general way and thus mutations of both genes can be expected to have pleiotropic effects. Experiments described in the present invention shed further light on the molecular pathogenesis of fibroids providing data concerning coexistence of MED12 mutations with other known mutations in fibroids and in particular with those affecting the HMGA genes; see, e.g., Example 2 and Table 1.

Moreover, the present invention provides data indicating to which extent these mutations occur in other benign or malignant tumors as well; see, e.g., Example 5. Of note, uterine fibroids belong to a large group of benign tumors frequently showing chromosomal rearrangements of the loci of the two genes encoding HMGA proteins (HMGA1, HMGA2) as well as cases with an apparently normal karyotype. Examples of other tumors with these abnormalities are endometrial polyps [16-18] and lipomas [8,19]. Thus, the important question is addressed whether in these tumors the “fibroid-type” MED12 mutations can be found as well.

Experimental data obtained in experiments performed within the scope of the present invention addressing these questions and arising from the analysis of a series of 80 cytogenetically characterized fibroids as well as 21 endometrial polyps for MED12 mutations of the “fibroid-type” [11] are described; see, e.g., Example 1 and Table 1. Due to the experimental results obtained in accordance with the present invention, novel therapeutic targets such as the Wnt4 gene as well as interesting markers for the diagnosis and prediction of the course of the disease, e.g., different MED 12 mutations and the Wnt4 gene, have been identified and will be described in detail hereinbelow.

### IV. Wnt4 Inhibitors

Preliminary data obtained in accordance with the present invention concerning gene expression alterations in several fibroid samples indicate enhanced Wnt4 expression playing a key role in genesis of gynecological tumors; see, e.g., Example 4 and FIG. 3. Therefore, the present invention generally relates to a Wnt4 inhibitor for use in the treatment of a benign or malignant gynaecological tumor, for example, wherein the tumor is selected from the group consisting of endometrial polyps, endometriosis, adenomyosis, leiomyosarcomas of the uterus, aggressive angiomyxomas, endometrial carcinomas and Müllerian mixed tumors.

In a particularly preferred embodiment of the present invention the Wnt4 inhibitor is used for the treatment of uterine leiomyoma (UL)

The main aim of the use of Wnt4 inhibitors according to the present invention is to disturb or to inhibit the cell signalling permitted by Wnt4 activity thereby reducing the growth and proliferative potential of cells from gynecological tumors. Furthermore, generally all Wnt4 inhibitors can be used in a way to reduce or to disrupt cell signalling dependent of Wnt4 in aberrantly growing mesenchymal stem cells and their descendants, e.g., leiomyoma cells. There are several possible modes of action by which these effects can be achieved in respect of the present invention, wherein the Wnt4 inhibitors can be used besides for a direct inhibition of the interaction of Wnt4 with the members of the frizzled family of seven transmembrane receptors and/or members of Low Density Lipoprotein Receptor-related Protein (LRP) family, e.g. LRP-5 or LRP-6 involved in reception of Wnt-signalling as co-receptors for a reduction of Wnt4 activity by disturbing or inhibiting of one or more processes such as the following: Wnt4 gene expression, splicing of the Wnt4-mRNA, maturing of the Wnt4-mRNA, transport of the mRNA out of the nucleus, translation of Wnt4 mRNA, transport of the Wnt4-protein through the cell, its secretion from the signalling cell and/or interfering with the Wnt-signal transduction from the receptor at the cell membrane to the nucleus by interfering with the molecules involved in the signal transduction such as, e.g., Axin, GSK-3 (glycogen syntase kinase-3) or beta-catenin. Generally, Wnt4 inhibitors of the present invention include but are not limited to “antigen binding molecules” binding with a specific binding affinity its corresponding target molecule, e.g., an antigen of interest or a nucleic acid of interest such as the Wnt4 protein and (pre) mRNA encoding it or corresponding genomic DNA. An “antigen binding molecule” is any molecule that has at least an affinity of 10⁵ l/mol for its target molecule. The antigen-binding molecule, i.e. Wnt4 inhibitor of the present invention preferably has an affinity of 10⁶ of 10⁷, or also preferred at least 10⁸ or 10⁹, or more preferred at least 10¹⁰, 10¹¹ or 10¹² l/mol for its target molecule. Preferably the antigen-binding molecule specifically binds to the target of interest. As the skilled artisan will appreciate, the term specific is used to indicate that other biomolecules present in the sample do not significantly bind to the antigen-binding molecule. Preferably, the level of binding to a biomolecule other than the target molecule results in a binding affinity which is at most only 10% or less, only 5% or less only 2% or less or only 1% or less of the affinity to the target molecule, respectively. A preferred specific binding agent will fulfill both the above minimum criteria for affinity as well as for specificity.

Inhibitors of the Wnt-signalling pathway are known in the art. For example, the non-steroidal anti-inflammatory compound sulindac (CAS Registry No. 38194-50-2; described in U.S. Pat. No. 3,654,349) is an exemplary Wnt-signalling pathway inhibitor. In particular, sulindac inhibits β-catenin/LCF-regulated transcription of target genes [50-52]. Inhibition of the Wnt-signalling pathway by other inhibitors such as antibodies, aptamers or small molecules has been described as well in, e.g., international applications WO 2011/103426 and WO 2010/146055 and [53].

Despite of the presence and usage of several distinct Wnt ligands in one organism, most of the other components of the Wnt-pathway are highly conserved and used for the transduction of signals elicited by said several Wnt ligands. Thus, the above described methods can be used at least in an analogous manner for Wnt4 inhibition. In one embodiment of the present invention the Wnt4 inhibitor is selected from the group consisting of small molecules, antibodies, antigen-binding antibody fragments, aptamers, spiegelmers, siRNA and miRNA.

### V. Antibodies and Antigen-Binding Fragments Thereof

One class of molecules which can be used according to the present invention as a Wnt4 inhibitor are antibodies and antigen-binding fragments thereof. Methods for producing an antibody, in particular a monoclonal antibody in hybridoma cells, for example a human antibody are known in the art and are described, e.g., in Goding, “Monoclonal Antibodies: Principles and Practice”, Academic Press, pp 59-103 (1986). Methods for producing a chimeric antibody, murinized antibody, single-chain antibody, Fab-fragment, bi-specific antibody, fusion antibody, labeled antibody or an analog of any one of those are known as well to the person skilled in the art and are described, e.g., in Harlow and Lane “Antibodies, A Laboratory Manual”, CSH Press, Cold Spring Harbor (1988). The production of chimeric antibodies is described, for example, in international application WO89/09622. Methods for the production of humanized antibodies are described in, e.g., European application EP-A1 0 239 400 and international application WO90/07861. Further sources of antibodies to be utilized in accordance with the present invention are so-called xenogeneic antibodies. The general principle for the production of xenogeneic antibodies such as human-like antibodies in mice is described in, e.g., international applications WO91/10741, WO94/02602, WO96/34096 and WO 96/33735. As discussed above, the antibody of the invention may exist in a variety of forms besides complete antibodies; including antigen-binding antibody fragments, for example, Fv, Fab and F(ab)2, as well as in single chains; see e.g. international application WO88/09344.

### VI. Gene Inhibition and Molecules Used Therefor

Besides of antibodies and fragments thereof, which can be used according to the present invention to lower Wnt4 protein levels, expression of genes or levels of specific proteins in cells or organs can be reduced as well by techniques using antisense molecules, for example. “Antisense molecules” or “antisense reagents” can, in the present context, be any molecule that hybridizes by a sequence specific base pairing to a complementary DNA and/or RNA sequence. In the context of this invention, “hybridization” means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds.

It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid nonspecific binding of the antisense compound to non-target sequences under conditions in which specific binding is desired, i.e. under physiological conditions in the case of in vivo assays, and in the case of in vitro assays, under conditions in which the assays are performed. Typical “antisense molecules” or “antisense reagents” are any oligonucleotide, such as DNA, RNA, any peptide nucleic acid, any other nucleic acid derivative, or mimic and/or derivative thereof. The target sequence is not restricted to the “sense” or “coding” strand of mRNA, although this is often the target. According to the present invention “antisense molecules,” or “antisense constructs” can be employed which are used interchangeably in the present text. In one embodiment of the present invention the use of oligonucleotides, for use in modulating the function of nucleic acid molecules encoding genes, in particular of the Wnt4 gene is addressed. This is accomplished by providing antisense compounds which specifically hybridize with one or more nucleic acids encoding a target gene, such as the Wnt4 gene.

As used herein, the term “target nucleic acid” encompasses a DNA encoding said gene, and/or an RNA (including pre-mRNA and mRNA) transcribed from such DNA. The specific hybridization of an oligomeric compound with its target nucleic acid interferes with the normal function of the nucleic acid. This modulation of function of a target nucleic acid by compounds which specifically hybridize to it is generally referred to as “antisense” (when the target is RNA) or “antigene” (when the target is DNA). The functions of DNA to be interfered with include replication and transcription. This effect is referred to as “antigene”. Such interactions may occur by binding of the “antigene” molecule to the DNA double-helix as a third strand in its major groove forming a structure also known as “triplex DNA” or “triple helix DNA” (Frank-Kamenetskii, Annu. Rev. of Biochem. 64 (1995), 65-95; Rusling et al., Nucleic Acids Res. 33 (2005), 3025-3032). The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA and is referred to as “antisense”. However, the distinction between “antisense” and “antigene” is not absolute.

The overall effect of such interferences with target nucleic acid function is a specific modulation of the expression of said essential gene. In the context of the present invention, “modulation” means either an increase (stimulation) or a decrease (inhibition) in the expression of a gene. In the context of the present invention, in particular concerning modulation of Wnt4, inhibition is the preferred form of modulation of gene expression.

In the present invention, antisense molecules can be selected from the group consisting of oligonucleotides, oligonucleotide analogues, oligonucleotide mimics, such as for example PNA, locked nucleic acids (LNA), phosphorothioate, 2′-methoxy-, 2′-methoxyethoxy-, morpholino, phosphoramidate oligonucleotides or the like. In the present invention, antigene molecules can furthermore be selected from the group consisting of triplex forming or strand invading oligonucleotides, oligonucleotide analogues, oligonucleotide mimics, such as for example PNA, locked nucleic acids (LNA), phosphorothioate, 2′-methoxy-, 2′-methoxyetyhoxy-, morpholino, phosphoramidate oligonucleotides or DNA minor groove binding polyamides (oligo pyrroles/imidazoles etc.) as described (Gottesfeld et al., Gene Expr. 9 (2000), 77-91; Dervan and Bürli, Curr. Opin. Chem. Biol. 3 (1999), 688-693) and the like.

The term “oligonucleotide(s)” refers to an oligomer or polymer of ribonucleic acid (RNA) or deoxyribonucleic acid (DNA) or mimetics thereof. This term includes oligonucleotides composed of naturally-occurring nucleobases, sugars and covalent internucleoside (backbone) linkages as well as oligonucleotides having non-naturally occurring nucleobases, sugars and covalent internucleoside (backbone) linkages which function similarly or combinations thereof. Such modified or substituted oligonucleotides are often preferred over native forms because of desirable properties such as, for example, enhanced cellular uptake, enhanced affinity for nucleic acid target and increased stability in the presence of nucleases and other enzymes, and are in the present context described by the terms “oligonucleotide analogues” or “oligonucleotide mimics”.

The antisense compounds in accordance with this invention preferably comprise from 7 to 80 nucleobase units, preferably not more than 30 nucleobase units to avoid an interferon response (Manche et al., Mol. Cell. Biol. 12(1992), 5238-5248). The term “nucleobase units” is used in the present text to describe both the number of nucleotides in an oligonucleotide and the number of nucleobase-carrying monomers of an oligonucleotide mimetic. Particularly preferred antisense compounds are antisense oligonucleotides, even more preferably those comprising from 14 to 29 nucleobases. Most preferred are short RNA based antisense oligonucleotides comprising around 20 nucleobases, i.e. from 18 to 26 nucleobases, of two particular molecular classes, either single stranded (miRNA) or double stranded (siRNA).

Unmodified, naked antisense molecules were reported to be internalized poorly by cells, whether or not they are negatively charged (Grey et al., Biochem. Pharmacol. 53 (1997). 1465-1476, Stein et al., Biochemistry 32 (1993), 4855-4861. Bennet et al., Mol. Pharmacol. 41 (1992), 1023-1033). Therefore, the oligonucleotides may be modified or used in compositions with other agents such as lipid carriers (Fattal et al., Adv. Drug Deliv. Rev. 56 (2004), 931-946), microparticles (Khan et al., J. Drug Target 12 (2004), 393-404) or by covalent conjugation to cell-penetrating peptides (CPP) allowing translocation of the antisense molecules through the cell membrane; see Lysik and Wu-Pong, J. Pharm. Sci. 92 (2003), 1559-1573 for an review.

As used herein, the term “aptamer” refers to a DNA or RNA molecule that has been selected from random pools based on their ability to bind other molecules with high affinity specificity based on non-Watson and Crick interactions with the target molecule (see, e.g., Cox and Ellington, Bioorg. Med. Chem. 9 (2001), 2525-2531; Lee et al., Nuc. Acids Res. 32 (2004), D95-D100). In accordance with the present invention aptamers can be selected which bind molecules such as nucleic acids or proteins.

The peptides and aptamers of the present invention are synthesized by any suitable method. For example, targeting peptides and aptamers of the present invention can be chemically synthesized by solid phase peptide synthesis. Techniques for solid phase synthesis are described, for example, by Barany and Merrifield (1979) Solid-Phase Peptide Synthesis; pp. 1-284 in The Peptides: Analysis, Synthesis, Biology, (Gross. and Meinehofer, eds.), Academic, New York, Vol. 2, Special Methods in Peptide Synthesis, Part A.; Merrifield, J. Am. Chem. Soc, 85 (1963), 2149-2154; and Stewart and Young (1984) Solid Phase Peptide Synthesis, 2nd ed. Pierce Chem. Co., Rockford, Ill.

Spiegelmers are nucleic acids comprising a number of L-nucleotides which show binding activities towards a target or a part thereof. The basic method of Spiegelmer generation is subject to the international patent application WO 1998/008856 the disclosure of which is incorporated herein by reference. Basically, this method relies on the so-called SELEX technique as described, e.g., in U.S. Pat. No. 5,475,096. The method uses combinatorial DNA or RNA libraries comprising a randomised stretch of about 10 to about 100 nucleotides which are flanked by two primer binding regions at the 5′ and 3′ end. The generation of such combinatorial libraries is, for example, described in Conrad et al., Methods Enzymol., 267 (1996), 336-367. Such a chemically synthesized single-stranded DNA library may be transferred into a double-stranded library via polymerase chain reaction.

Such a library may already be used for selection purpose. The selection occurs such that the, typically single-stranded, library is contacted with a target molecule and the binding elements of the library are then amplified. By repeating these steps several times oligonucleotide molecules may be generated having a significant binding activity towards the target used.

Spiegelmers, as said above, are actually L-polynucleotides which are generated such that D-polynucleotides are selected against a target molecule which is present in its non-naturally occurring enantiomer, and the nucleic acid binding thereto is then synthesized using L-nucleotides creating the L-polynucleotide, which is the spiegelmer. This L-polynucleotide is capable of binding to the target molecule in its naturally occurring form. In case the target is a protein or peptide the non-naturally occurring enantiomer is the D-protein/peptide and the naturally occurring enantiomer is the L-protein/peptide. In accordance with the present invention spiegelmers can be used which bind molecules such as proteins, peptides or nucleic acids.

### VII. Methods of the Present Invention

Preliminary results obtained in accordance with the present invention (see, e.g., Example 4 and FIG. 3) indicate a surprising correlation between specific base substitutions in the MED12-locus and Wnt4 expression. Therefore, in one embodiment of the present invention a method is provided to determine the response potential of a tumor as defined hereinabove and below to a treatment with a Wnt4 inhibitor, comprising:
(a) detecting at least one MED12-mutation affecting the sequence CAAGGT (corresponding to bases 326 to 331 of the MED12-mRNA-sequence of SEQ ID NO: 1) encoding codons 43 to 44 of the MED12-gene, in a test sample derived from a patient, wherein the presence of said at least one MED12-mutation is indicative for a tumor responsive to treatment by Wnt4 inhibitors; and/or
(b) determining Wnt4 expression in a test sample derived from a patient, wherein an enhanced expression compared to a control sample is indicative for a tumor responsive to treatment by Wnt4 inhibitors.

The cDNA-Sequence of MED12 with underlined nucleotides c.127 to c.132 is enlisted in Table 2 further below.

Further preliminary results obtained in accordance with the present invention (see, e.g., in Example 3 and FIG. 2) indicate a close relationship between the occurrence of specific point mutations in the MED12 gene locus and the growth potential of the tumors composed of the mutated cells. Thus, in one embodiment a method for detection of at least one MED12-mutation as defined hereinabove for use in determining the growth potential of a tumor as defined hereinabove comprising detecting at least one MED12-mutation in a test sample derived from a patient, wherein c.130 or c.131G>A transitions at codons 43 or 44 of the MED12-gene are indicative of a higher growth potential of the tumor compared to a tumor comprising different MED12-mutations at codons 43 or 44 is provided.

Several reports have shown that Wnt4 signaling may contribute directly or indirectly to initiation or progression of tumors due to the regulation of Wnt4 expression by several tumor suppressors including the classic tumor suppressor p53 gene family members p63 and p73, the Wilms' tumor suppressor WT1, and the cyclin/CDK inhibitor p21 [41; 45-47]. In particular, published data suggest Wnt4 involvement in the proliferation and survival of the pituitary adenoma cells [48] and possible involvement in development of prostate tumors due to an autoregulatory negative feedback-loop between EAF (ELL-associated factor) family members, EAF1 and EAF2/U19 which play a role in cancer and embryogenesis and Wnt4.

Therefore, in another embodiment the present invention provides a method for diagnosing a pituitary tumor, a prostate tumor or a prostate hyperplasia comprising detecting MED12 mutations in a test sample derived from the respective pituitary gland or prostate.

In the majority of cases, there is no great difficulty to distinguish between benign and malignant smooth muscle tumors. Nevertheless, in rare cases the exact diagnosis is difficult and thus the development of strategies for the differential diagnosis in these cases still remains an unmet challenge. In this context, due to the preliminary experimental results obtained in accordance with the present invention, new methods and kits are provided herein allowing differential diagnosis indicative for the malignancy of an analysed tumor sample.

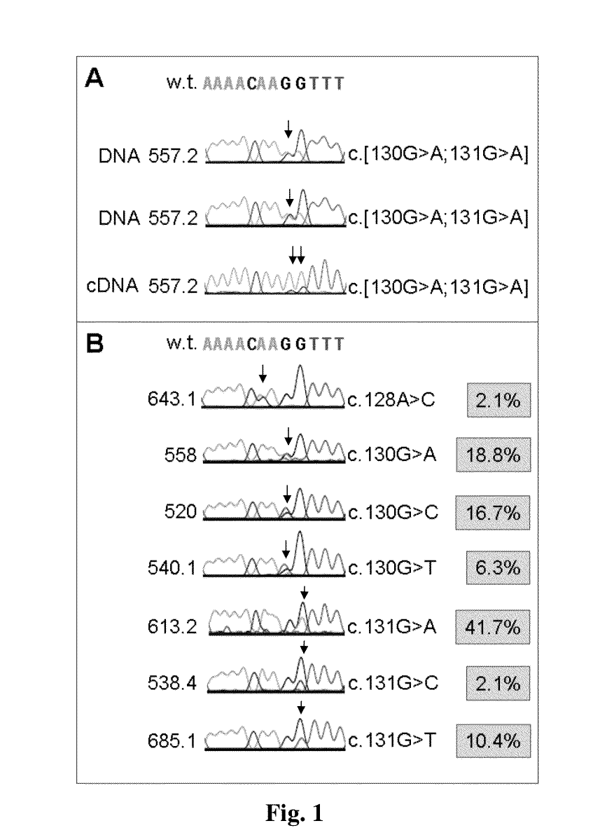

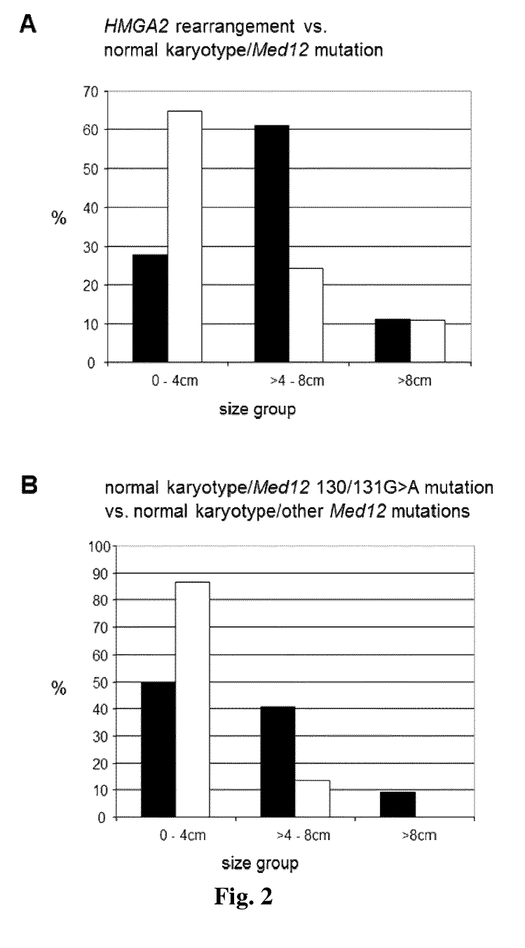

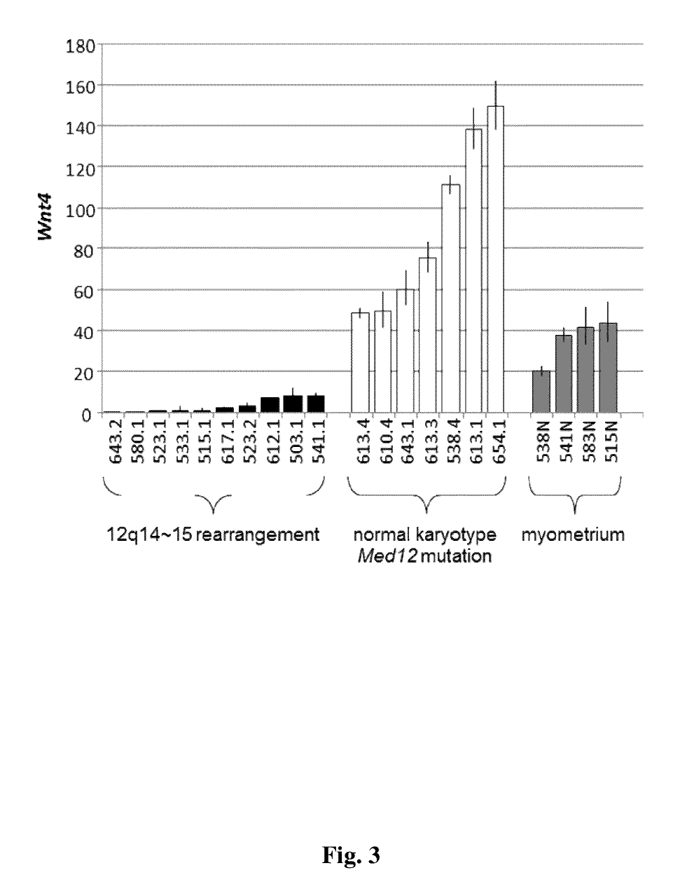

According to the experimental data provided and discussed herein, MED12 mutations and rearrangements of the gene encoding high mobility group protein AT-hook 2 (HMGA2) occur in apparently mutually exclusive uterine leiomyomas types. However, it is not clear yet whether MED12 mutations occur in malignant uterine tumors as well. Surprisingly, the experiments performed within the scope of the present invention show that MED12 mutations are very rare in malignant uterine tumors (see, Example 6 and Tab.3). Thus, in accordance with the experimental results provided within the scope of the present invention and since former experiments have shown that HMGI-2 (formerly HMGI-C) expression levels in normal differentiated tissues are very much lower than in malignant tissues [see, e.g., European patent application EP 072 748 7 A1 and citations 54-56] a method to distinguish between benign and malignant smooth muscle tumors of the uterus is provided herein as depicted in an extremely schematic manner in FIG. 4. By using these algorithms or a different combination of their parameters (including as well analysis of potential HMGA1 rearrangements where the proposed algorithm does not lead to informative results) a skilled person will be able to unambiguously distinguish between benign and malignant smooth muscle tumors.

In this respect, the present invention provides a method for differential diagnosis of uterine smooth muscle tumors comprising:
(a) detection of a mutation in the MED12 gene and its expression; and/or
(b) determination of expression of the gene encoding high mobility group protein AT-hook 2 (HMGA2) and/or of rearrangements of the HMGA1 and/or HMGA2 gene locus in a test sample from a patient; wherein:
(i) increased and/or ectopic HMGA2 expression, absence of a MED12 mutation, and absence of rearrangements of the HMGA2 gene locus are indicative for a malignant smooth muscle tumor;
(ii) increased and/or ectopic HMGA2 expression, and presence of rearrangements of the HMGA2 gene locus are indicative for a benign smooth muscle tumor;
(iii) presence of a MED12 mutation, normal HMGA2 and MED12 expression and absence of rearrangements of the HMGA2 gene locus are indicative for a benign smooth muscle tumor;
(iv) presence of rearrangements of the HMGA1 gene locus, normal HMGA2 expression, absence of a MED12 mutation and absence of rearrangements of the HMGA2 gene locus are indicative for a benign smooth muscle tumor;
(v) presence of a MED12 mutation, normal HMGA2 expression, not detectable MED12 expression and absence of rearrangements of the HMGA2 gene locus are indicative for a malignant smooth muscle tumor; and
(vi) increased and/or ectopic HMGA2 expression and presence of a MED12 mutation are indicative for a malignant smooth muscle tumor.

Preliminary experimental results provided within the scope of the present invention indicate that mutations of the MED12 gene in leiomyomas may preferentially be found in sequence regions encoding codons 43 and 44. Therefore, in one embodiment of the present invention the method for differential diagnosis is provided, wherein the MED12 gene is analyzed for presence of a mutation affecting the sequence CAAGGT (corresponding to bases 326 to 331 of the MED12-mRNA-sequence of SEQ ID NO: 1) encoding codons 43 to 44.

In a preferred embodiment of the present invention, the method for differential diagnosis is provided, wherein the malignant smooth muscle tumor is leiomyosarcoma and the benign smooth muscle tumor is leiomyoma.

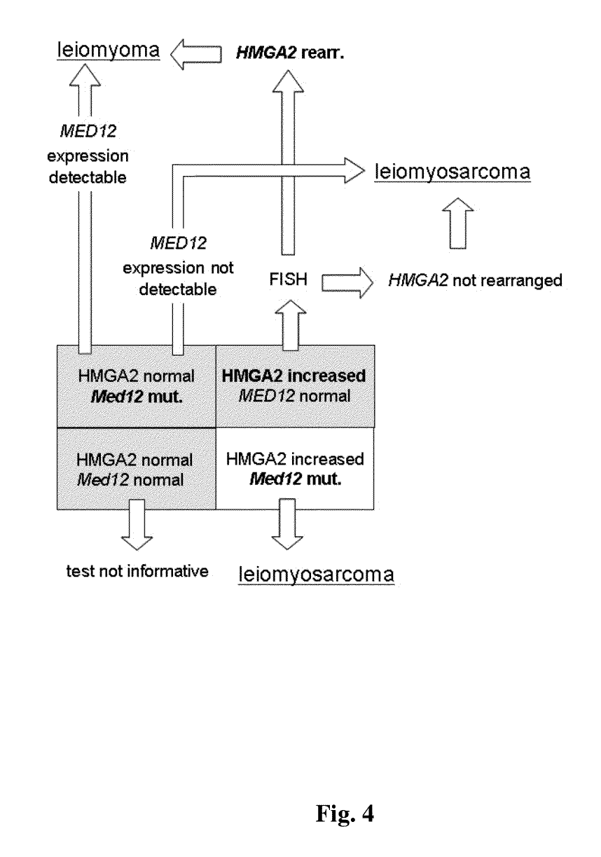

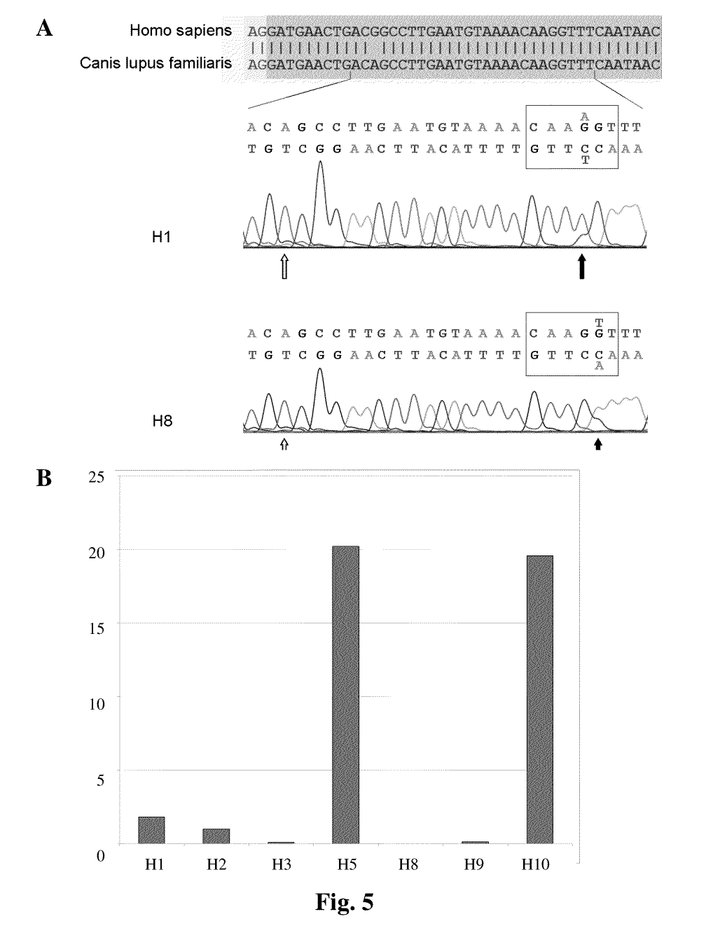

Due to various similarities in biology and presentation of human and canine cancers, dogs are used besides rodents as a further animal model for therapeutic and preclinical studies and offer additional means to elucidate the pathogenesis of tumor formation, study the effects of hormones and agents on the development and growth of these tumors as well as to test potential therapeutic modalities. In this respect, preliminary experimental results provided herein show that in dogs the same main genetic groups of uterine leiomyomas exist as found in humans, i.e. occurrence of MED12 mutations, rearrangements and/or overexpression of HMGA genes (see, e.g., Example 7 and FIG. 5). It is prudent thus, to conclude that both types of mutations are a general phenomenon characterizing subtypes of uterine leiomyomas in mammals.

Therefore, in a further embodiment the present invention provides a method for identification of suitable mammalian models for different types of smooth muscle tumors comprising the method for differential diagnosis as defined hereinabove, wherein the presence of a MED12 mutation, HMGA2 expression and/or presence of rearrangements of the HMGA2 and/or HMGA1 gene locus are analyzed in respect of homologues of the human MED12, HMGA1 and HMGA2 genes in a test sample of the respective mammal.

As used herein, “orthologues” are separate occurrences of the same gene in multiple species. The separate occurrences have similar, albeit nonidentical, amino acid sequences, the degree of sequence similarity depending, in part, upon the evolutionary distance of the species from a common ancestor having the same gene. As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences. Normally, orthologs retain the same function in the course of evolution [57]. Paralogues often retain the same or a similar function.

The term “homologues”, as used herein, is generic to “orthologues” and “paralogues”.

### VIII. Kits

In one embodiment the present invention relates to a kit useful in a method as defined hereinabove, comprising one or more reagents for detecting the MED12-mutations. In one embodiment the above-mentioned kit is provided, wherein the reagents comprise an antibody or a nucleic acid.

In a further embodiment the above-mentioned kit is provided, comprising primers for the amplification of a fragment of the genomic template DNA region comprising the MED12 locus, and/or for amplification of a target cDNA-fragment generated from a MED12-mRNA and/or for sequencing of said amplified fragments.

Furthermore, in one embodiment the above-mentioned kit is provided comprising primers for the quantification of Wnt4 expression in a test sample.

In a further embodiment, the above-mentioned kit is provided further comprising reagents for the quantification of HMGA2 expression, for detection of HMGA2 expression and/or of rearrangements of the HMGA2 and/or HMGA1 gene locus in a test sample.

The examples which follow further illustrate the invention, but should not be construed as to limit the scope of the invention in any way. Detailed descriptions of conventional methods, such as those employed herein can be found in the cited literature; see also “The Merck Manual of Diagnosis and Therapy” Seventeenth Ed. edited by Beers and Berkow (Merck & Co., Inc. 2003). The practice of the present invention will employ, unless otherwise indicated, conventional techniques of cell biology, cell culture, molecular biology, transgenic biology, microbiology, recombinant DNA, and immunology, which are within the skill of the art.

Suitable regimens for therapeutic administration and methods for preparing pharmaceutical compositions of the invention are within the skill in the art, for example as described in Remington's Pharmaceutical Science, 17th ed., Mack Publishing Company, Easton, Pa. (1985) and update version Remington: The Science and Practice of Pharmacy (2000) by the University of Sciences in Philadelphia, ISBN 0-683-306472, the entire disclosure of both documents which is incorporated herein by reference.

Methods in molecular genetics and genetic engineering are described generally in the current editions of Molecular Cloning: A Laboratory Manual, (Sambrook et al., (1989) Molecular Cloning: A Laboratory Manual, 2nd ed., Cold Spring Harbor Laboratory Press); DNA Cloning, Volumes I and II (Glover ed., 1985); Oligonucleotide Synthesis (Gait ed., 1984); Nucleic Acid Hybridization (Hames and Higgins eds. 1984); Transcription And Translation (Hames and Higgins eds. 1984); Culture Of Animal Cells (Freshney and Alan, Liss, Inc., 1987); Gene Transfer Vectors for Mammalian Cells (Miller and Calos, eds.); Current Protocols in Molecular Biology and Short Protocols in Molecular Biology, 3rd Edition (Ausubel et al., eds.); and Recombinant DNA Methodology (Wu, ed., Academic Press). Gene Transfer Vectors For Mammalian Cells (Miller and Calos, eds., 1987, Cold Spring Harbor Laboratory); Methods In Enzymology, Vols. 154 and 155 (Wu et al., eds); Immobilized Cells And Enzymes (IRL Press, 1986); Perbal, A Practical Guide To Molecular Cloning (1984); the treatise, Methods In Enzymology (Academic Press, Inc., N Y); Immunochemical Methods In Cell And Molecular Biology (Mayer and Walker, eds., Academic Press, London, 1987); Handbook Of Experimental Immunology, Volumes I-IV (Weir and Blackwell, eds., 1986). Reagents, cloning vectors, and kits for genetic manipulation referred to in this disclosure are available from commercial vendors such as BioRad, Stratagene, Invitrogen, and Clontech. General techniques in cell culture and media collection are outlined in Large Scale Mammalian Cell Culture (Hu et al., Curr. Opin. Biotechnol. 8 (1997), 148); Serum-free Media (Kitano, Biotechnology 17 (1991), 73); Large Scale Mammalian Cell Culture (Curr. Opin. Biotechnol. 2 (1991), 375); and Suspension Culture of Mammalian Cells (Birch et al., Bioprocess Technol. 19 (1990), 251); Extracting information from cDNA arrays, Herzel et al., CHAOS 11 (2001), 98-107. Several documents are cited throughout the text of this specification. Full bibliographic citations may be found at the end of the Examples immediately preceding the Tables and the Claims. The contents of all cited references (including literature references, issued patents, published patent applications as cited throughout this application and manufacturers specifications, instructions, etc) are hereby expressly incorporated by reference; however, there is no admission that any document cited is indeed prior art as to the present invention.

### EXAMPLES

### Material and Methods

### Tissue Samples

Samples of uterine leiomyomas and matching myometrium were taken during or directly after surgery, immediately frozen in liquid nitrogen, and stored at −80° C. for DNA and RNA isolation. For cell culture and karyotype analyses tumor samples were directly transferred to Hank's solution. The study was approved by the local ethics committee and prior to surgery, informed written consent was obtained from all patients. Samples of formalin-fixed paraffin-embedded tissue (FFPE-samples) of 21 endometrial polyps were used for DNA sequence analyses. All these samples were initially taken for diagnostic purposes and de-identified prior to their use in the present study.

### Cell Culture of Uterine Leiomyomas

From tissue samples stored in sterile Hank's solution cell cultures were set up as described previously [20]. Briefly, the samples were minced into small pieces and treated with collagenase. The dissociated cells were transferred into cell culture flasks and incubated in 5% CO₂ air at 37° C.

### Cytogenetic and Molecular Cytogenetic Studies of Uterine Leiomyomas

Chromosome analyses and fluorescence in situ hybridization (FISH) on slides prepared according to conventional cytogenetics were performed following routine techniques as described previously [20]. For FISH on tissue sections three BAC clones (RP11-745O10 (AC078927) and RP11-293H23 (AC012264) located distal (3′) and RP11-269K4 (AQ478964 and AZ516203) located proximal (5′) of HMGA2) were used as break-apart probe. Labelling was performed by nick translation (Abbott Molecular, Wiesbaden, Germany) either with SpectrumOrange-dUTP (RP11-745O10 and RP11-293H23) or SpectrumGreen-dUTP (RP11-269K4) (Abbott Molecular, Wiesbaden, Germany). Pretreatment of 4 μm tissue sections was performed as described previously for formalin-fixed, paraffin-embedded tissue sections [21] with a few modifications. Digestion with a pepsin ready-to-use solution (DCS, Hamburg, Germany) was performed at 37° C. for 2×45 min 15 μl of the break-apart probe (concentration 100 ng/10 μl) was used per slide. Co-denaturation was performed on a ThermoBrite (Abbott Molecular) for 5 min at 85° C. followed by overnight hybridization in a humidified chamber at 37° C. Post-hybridization was performed at 42° C. for 2 min in 0.4×SSC/0.3% NP-40. Interphase nuclei were counterstained with DAPI (0.75 μg/ml). Slides were examined with a Axioskop 2 plus fluorescence microscope (Carl Zeiss, Göttingen, Germany), images were captured with an high performance CCD-camera (Visitron Systems, Puchheim, Germany) and edited with FISH View (Applied Spectral Imaging, Migdal HaEmek, Israel). 100 non-overlapping nuclei from four different areas of the tumor were scored.

### DNA Isolation

DNA was isolated from frozen tissue samples by using the QIAamp DNA Mini Kit (Qiagen, Hilden, Germany) and DNA from formalin-fixed, paraffin embedded (FFPE) tissue samples was isolated using the QIAamp DNA FFPE Tissue Kit (Qiagen) using the QIACube (Qiagen) according to manufacturer's instructions.

### RNA Isolation

Total RNA from frozen tissue samples was isolated using a RNeasy Mini Kit (Qiagen) in a QIACube (Qiagen) according to manufacturer's instructions and DNase I digestion was performed.

cDNA-Synthesis

250 ng of total RNA were reverse transcribed with M-MLV reverse transcriptase (Invitrogen, Karlsruhe, Germany), RNase Out (Invitrogen), random hexamers and dNTPs according to the manufacturer's instructions. RNA was denatured at 65° C. for 5 min and subsequently kept on ice for 1 min After adding the enzyme to the RNA primer mixes, samples were incubated for 10 min at 25° C. to allow annealing of the random hexamers. Reverse transcription was performed at 37° C. for 50 min followed by inactivation of the reverse transcriptase at 70° C. for 15 min.

### PCR and Sequencing

For PCR amplifications 1,000 ng of genomic template DNA or 1,000 ng of previously synthesized cDNA-template were used, respectively. Primers used to amplify the desired PCR fragment of the genomic template DNA were 5′-CCC CTT CCC CTA AGG AAA AA-3′ (Forward 1; SEQ ID NO: 3) and 5′-ATG CTC ATC CCC AGA GAC AG-3′ (Reverse 1; SEQ ID NO: 4). For amplification of the target cDNA-fragment primers were 5′-CTT CGG GAT CTT GAG CTA CG-3′ (Forward 2; SEQ ID NO: 5) and 5′-ATG CTC ATC CCC AGA GAC AG-3′ (Reverse 1; SEQ ID NO: 4). Subsequently, PCR-products were separated by agarose gel-electrophoresis and the desired DNA-fragments/-bands were extracted by a QIAquick Gel Extraction Kit (Qiagen) using a QIACube (Qiagen) according to manufacturer's instructions. DNA-sequencing of the purified PCR-products was performed by GATC Biotech (GATC Biotech, Konstanz, Germany).

### Quantitative Real-Time PCR

Relative quantification of transcription levels was carried out by real-time PCR analyses using the Applied Biosystems 7300 Real-Time PCR system (Applied Biosystems, Darmstadt, Germany). For quantification of Wnt4 mRNA (Hs01573504_m1) a commercially available gene expression assay (Applied Biosystems) was used. HPRT served as endogenous control as described before [10].

### Cytogenetic and Gene Mutation Nomenclature

Cytogenetic nomenclature followed ISCN [22] and gene mutation on the DNA level are described according to [23].

### Statistical Analyses

The statistical significance of differences was assessed by the student's t test. In all comparisons, p<0.05 was considered statistically significant.

### Example 1

### Frequent Occurrence of Single MED12 Mutations in a Series of Uterine Leiomyomas Including One Tumor with Two Mutations

By PCR amplification and sequencing genomic DNA and cDNA samples from a total of 80 cytogenetically characterized uterine fibroids from 50 patients were analyzed for mutations of MED12 as recently described by Mäkinen and coworkers [11] which we shall refer herein as to “fibroid-type MED12 mutations”. Of the tumors investigated, 48 had an apparently normal karyotype without evidence for clonal chromosomal deviations after conventional cytogenetic examination based on a band resolution ranging from approximately 350 to 650 bands/haploid set. These latter tumors were randomly selected from a larger group of fibroids. 20 fibroids had an either simple or complex rearrangement of chromosomal region 12q14˜15 targeting the locus of high mobility group AT-hook 2 (HMGA2) leading to its significant upregulation. Of the remaining tumors, six had a clonal deletion or rearrangement of the long arm of chromosome 7 as the sole karyotypic abnormality, five showed rearrangements of chromosomal band 6p21˜23, and in one fibroid a clonal trisomy 12 was detected.

Of these 80 fibroids, 47 (58.8%) revealed “fibroid-type MED12 mutations”. All mutations were heterozygous and in all but one fibroid only one mutation was detected. The remaining exceptional tumor (no. 557.2, see table 1) revealed G>A transitions at nucleotides c.130 and c.131 which were confirmed by repeated DNA isolation as well as by cDNA sequencing (FIG. 1 A). Base exchanges of codon 44 at positions 130 and 131 were predominantly observed accounting for 46/48 mutations (95.8%) (Tab.1). Among these the most prevalent mutation was the c.131 G>A base substitution (41.7%), followed by the c.130 G>A (18.8%) and the c.130 G>C (16.7%) substitution. However, albeit at much lower frequencies at positions 130 and 131 all other possible base substitutions were found as well (FIG. 1 B). This confirms the data obtained by Mäkinen et al. [11] in that in none of the cases analyzed the mutations were found in the matching myometrium. Also, cDNA analyses revealed that the tumors predominantly expressed the mutated allele suggesting that only mutations of the active allele are biologically relevant in terms of tumor development.

### Example 2

### Mutations of MED12 are Strongly Associated with Fibroids not Displaying Primary Karyotypic Alterations and as a Rule Had Preceded Secondary Karyotypic Alterations

When differentiating according to the karyotype groups it turned out that MED12 mutations were found in 36/45 tumors with an apparently normal karyotype (80%) but in none of the nodules with 12q14˜15 rearrangements. In contrast, six fibroids were analyzed that showed clonal deletions or rearrangements of the long arm of chromosome 7 as the sole clonal karyotypic abnormality. Whereas in two of these cases the aberration was found in all metaphases analyzed, in the remaining four cases chromosomal mosaicisms with the presence of aberrant as well as normal metaphases were noted. MED12 mutations were found in four of these cases. Next five fibroids were checked with rearrangements of chromosomal band 6p21˜23. Because akin to the 12q14˜15 aberrations it is difficult to determine exactly the chromosome 6 breakpoint by conventional cytogenetics all five tumors were in addition checked by fluorescence in situ hybridization (FISH) for HMGA1 rearrangements and by qRT-PCR for the expression of HMGA1 mRNA. All five cases had shown HMGA1 rearrangements and clearly elevated levels of HMGA1 mRNA, respectively ([24] and unpublished data). DNA sequencing revealed a MED12 mutation in three of them. The 6p21˜23 rearrangements were restricted to a clear minority of metaphases in two of these cases but from the results of neither DNA nor cDNA sequencing the mutations seemed to be confined to a minority of the cells only and it can thus be concluded that the MED12 mutations had preceded the chromosomal aberration. A fibroid displaying mosaic trisomy 12 had a MED12 mutation as well.

### Example 3

### Significant Correlations Between the Genetic Alterations and Fibroid Size

Fibroids with chromosomal rearrangements [25] and more specifically 12q14˜15 rearrangements [26] have previously been reported to be larger than those with an apparently normal karyotype. In the present series on average tumors with an apparently normal karyotype and MED12 mutation were significantly smaller than those with HMGA2 rearrangement (4.0 cm vs. 6.0 cm) (p<0.01) (FIG. 2 A). Interestingly, among the tumors with apparently normal karyotype those with c.130 or c.131 G>A transitions were found to be larger than those with other base substitutions at codons 43 or 44 (4.5 cm vs. 3.0 cm) (p<0.05) (FIG. 2 B). In contrast, no differences of the patient's ages at the time of surgery were noted between any of these subgroups.

### Example 4

### The Activation of the Gene Encoding Wingless-Type MMTV Integration Site Family, Member 4 (Wnt4) Plays a Key Role in Tumorigenesis Driven by Mutant MED12

A comparative gene expression analysis of MED12-mutated fibroids and their matching myometrium as carried out by Mäkinen [11] has highlighted three pathways being significantly altered including the Wnt signalling pathway. There is ample evidence linking members of this pathway with Müllerian duct morphogenesis. Of these members Wnt4 and Wnt5a are known to be expressed in the mesenchyme of the Müllerian duct giving rise to the likely tissue of origin of uterine leiomyomas (for review see [27]). Interestingly, Wnt4 maps to chromosomal segment 1p36 which has been observed to be recurrently rearranged in uterine fibroids [15]. To check whether Wnt4 is a target gene in these cases qRT-PCR was used to quantify and compare the expression of Wnt4 between a group of fibroids with normal karyotype and MED12 mutation, those with HMGA2 rearrangements, and normal myometrium. The expression of Wnt4 mRNA in tumors with MED12 mutations and normal karyotype significantly exceeded that in fibroids with HMGA2 rearrangement (p<0.01) as well as that in normal myometrium (p<0.05)(FIG. 3).

### Example 5

### MED12 Mutations are Rare in Endometrial Polyps and Seem to be Confined to Adenomyomatous Lesions

Uterine fibroids and endometrial polyps can have normal karyotypes as well as structural chromosomal aberrations affecting the loci of the human HMGA genes. Thus, next it was checked whether endometrial polyps as well might have the fibroid-type MED12 mutations. For this analysis, FFPE samples from 21 endometrial polyps have been investigated. With one exception of an atypical polypoid adenomyoma (syn.: adenomyomatous polyp), all other lesions histologically appeared to be simple glandular or fibrocystic polyps. DNA sequencing revealed MED12 mutations in two of these lesions. In the adenomyomatous polyp occurring in a 66 year old woman a heterozygous c.131G>A transition, i.e. the most frequent type of MED12 mutations in fibroids was found. A HMGA2 rearrangement was excluded by FISH. Histologically, the tumor showed irregular endometrioid-type glands embedded in a smooth muscle/fibromyomatous stroma. Microdissection followed by DNA-analysis showed that the mutation was not confined to a particular area of the polyp (data not shown). In a second tumor evidence for a MED12 mutation (c.130G>T) was found but after microdissection this turned out to have resulted from a small leimyoma present in the sample as well. Therefore, MED12 mutations seem to be rare findings in endometrial polyps probably confined to the rare adenomatous type.

### Example 6

### MED12 Mutations are Absent in Malignant Uterine Tumors

Tissue samples from a total of 50 malignant uterine tumors have been analyzed for MED12 mutations, by methods as described in the Materials and Methods section and in Example 1, supra. In particular, after DNA isolation from frozen tissue samples, PCR amplification and sequencing of genomic DNA and cDNA have been performed as indicated in detail above. A commercially available gene expression assay (Applied Biosystems) was used for quantification of human MED12 mRNA (Hs00192801_m1). HPRT served as endogenous control.

Investigated malignant tumor types, numbers of investigated cases and results of the analysis are indicated in Tab. 3 below. Surprisingly, the results as shown herein indicate that MED12 mutations are preferentially occurring in benign uterine tumors and are rare in their malignant counterparts.

TABLE 3
MED12 mutations are rare in malignant uterine tumors

| | | lesions positive for |
|---|---|---|
| | number of cases | leiomyoma-like |
| tumor type | investigated | MED12 mutations |
| malignant Muellerian | 11 | 0 |
| mixed tumors | | |
| leiomyosarcomas | 34 | 1 (no elevated level of |
| | | HMGA2 expression) |
| squamous cell carcinomas | 5 | 0 |

### Example 7

### Animal Models for Human Uterine Leiomyomas

To test the possibility that leiomyoma-like genetical aberrations found in humans may be the cause of similar tumors in other mammals, tissue samples from canine uterine leiomyomas have been analysed for presence of mutations in canine homologues of the MED12 and for the expression of the HMGA2 gene.

### PCR and Sequencing:

Primers used to amplify the desired PCR fragment of the canine template DNA as well as the canine target cDNA-fragment were 5′-GAT GAA CTG ACA GCC TTG AAT G-3′ (Forward 3; SEQ ID NO: 6) and 5′-CTT GGC AGG ATT GAA GTT GAC-3′ (Reverse 2; SEQ ID NO: 7). Subsequently, PCR-products were separated by agarose gel-electrophoresis and the desired DNA-fragments/-bands were extracted by a QIAquick Gel Extraction Kit (Qiagen) using a QIACube (Qiagen) according to manufacturer's instructions. DNA-sequencing of the purified PCR-products was performed by GATC Biotech (GATC Biotech, Konstanz, Germany).

### Quantitative Real-Time PCR:

Relative quantification of transcription levels was carried out by real-time PCR analyses using the Applied Biosystems 7300 Real-Time PCR system (Applied Biosystems, Darmstadt, Germany). For quantification of human HMGA2 mRNA (Hs00171569_m1; HMGA2 exons 1-2) a commercially available gene expression assay (Applied Biosystems) was used. HPRT served as endogenous control. Primers and probe used to amplify canine HMGA2 were 5′-AGT CCC TCC AAA GCA GCT CAA AAG-3′ (forward), 5′-GCC ATT TCC TAG GTC TGC CTC-3′ (reverse) and 5′-6-Fam-GAA GCC ACT GGA GAA AAA CGG CCA-TAMRA-3′ (probe).

Experiments performed in accordance with the present invention in dogs have shown the same main genetic groups of uterine leiomyomas as found in humans existing in dogs as well. In particular, DNA-sequencing of canine vaginal leiomyomas from dogs has shown leiomyoma-like mutations and occurrence of heterozygous MED12 mutations in canine MED12 gene (see FIG. 5A for sequencing results of H1 and H8). Furthermore, gene expression analysis (real-time RT-PCR) revealed two groups of canine leiomyomas characterized by high and low expression of HMGA2 mRNA. No MED12 mutations have been detected in samples from leiomyomas H5 and H10 showing an increased HMGA2 expression (FIG. 5B).

Thus, in accordance with the above indicated results a method is provided for distinguishing between benign and malignant smooth muscle tumors of the uterus.

### Discussion

Uterine leiomyomas are the most common gynaecological tumors and can even be considered the most frequent clinical relevant human tumors at all. Although ample epidemiologic data on this tumor are available (see, e.g., [28,29]) it is still reasonable to ask why “we know so little but could learn so much” [30]. The monoclonal origin of fibroids [3,5,6] suggests that mutations are the basis of these highly frequent tumors. Accordingly, they belong to the first benign tumors where recurrent cytogenetic deviations have been described [31,32]. Clonal chromosomal abnormalities can be found in roughly 20% of the fibroids [15]. Of these, 12q14˜15 rearrangements and deletions of part of the long arm of chromosome 7 represent the most frequent aberrations. Nevertheless, in the majority of fibroids no cytogenetic alterations can be observed. Moreover, a recent attempt [33] using a genome-wide analysis to detect possible loss of heterozygosity and copy number amplification in 37 leiomyomas revealed that copy number amplifications are infrequent events and generally do not determine clinical and histologic characteristics of the fibroids. Thus, there is no evidence for the existence of either genetic imbalances at the known loci or at other loci that may have escaped detection by means of conventional cytogenetics and might allow to identify target genes by positional cloning. In an alternative attempt, Mäkinen et al. [11] have used exome-sequencing resulting in the identification of apparently specific mutations of the mediator subcomplex 12 gene (MED12). Their clear predominance in the group of fibroids with an apparently normal karyotype and its absence in the tumors with 12q14˜15 rearrangements as both revealed by the experiments underlying the present invention are striking and fit with the larger size of fibroids without MED12 mutations compared to those without as observed by Mäkinen et al. [11] because the 12q14˜15 rearrangements can be expected to represent a large subset of the tumors without MED12 mutation. However, the results strongly suggest that MED12 mutations and HMGA2 activation due to chromosomal rearrangements pinpoint alternate pathways of myomagenesis. Taken together, both pathways might explain the genesis of roughly 85% of all fibroids. In contrast, other types of chromosomal aberrations as in particular the frequent deletions of a part of the long arm of chromosome 7 can apparently coexist with MED12 mutations as well as with rearrangements of HMGA2 confirming the results of earlier investigations [34] in that they represent secondary changes during the course of the disease that do not govern alternative lines of tumorigenesis. Of note, in contrast to HMGA2, rearrangements of HMGA1 (encoding the other gene of the human HMGA family of high mobility group proteins) fall within the category of genetic alterations that can coexist with MED12 mutations since in 4/5 tumors analyzed a chromosomal mosaicism was noted with the majority of cells having a normal female karyotype. On the other hand, from genomic DNA as well as cDNA sequencing no evidence was obtained that the MED12 mutations were restricted to a subpopulation of the tumor cells only. Of note, a difference in the growth potential mediated by the different possible mutations seems to exist that might explain the predominance of G>A transitions in clinically detectable fibroids.

While the association between cytogenetic subtypes and MED12 mutations has revealed novel insights into the different pathways of myomagenesis, a major challenge remains the understanding of the causal link between mutated Med12 and tumorigenesis. Data on the normal function of Med12 available, e.g., from hypomorphic mice and embryonic stem cells knocked-down for MED12 point to an essential role of Med12 in early mammalian development and the regulation of Nanog and Nanog target genes and in canonical Wnt and Wnt/PCP signalling [35,36]. The human CDK8 complex requires Med12 for its activity [27] and CDK8 is a known stimulus-specific positive coregulator of p53 target genes as in particular CDKN1a (p21) [37]. In turn CDKN1a is known to be upregulated by HMGA2 [10,38] and it is tempting to speculate that the mutated MED12 has lost its ability to positively regulate the CDKN1a locus thus protecting the cells from oncogene induced senescence. Nevertheless, from the results provided by the experiments underlying the present invention it can be excluded that HMGA2 activation and MED12 mutations cooperate synergistically in the development of fibroids because both groups obviously do not overlap, suggesting that they represent alternative pathways of tumor development mutually excluding each other. Interestingly, a comparative pathway analysis between eight mutation positive fibroids and their matching myometrium carried out by Mäkinen et al. [11] has highlighted three significantly altered pathways, i.e. focal adhesion, extra-cellular matrix receptor interaction and the Wnt signaling pathway. As to the latter, members of the Wnt family have been implicated in the development of tissues and organs derived from the Müllerian duct [27]. Within the canonical Wnt pathway the Wnt-ligands exert their effects by activation, i.e. translocation of beta-catenin from the cytoplasm to the nucleus (for review see [39]). In a mouse model constitutively expressing activated beta-catenin in the uterine mesenchyme, mesenchymal tumors leiomyoma-like lesions were found to develop with a 100% penetrance [40]. One might speculate that activation of beta-catenin by the Wnt pathway may be the mechanism by which MED12 mutations drive leiomyomagenesis. Interestingly, the present invention provides data and makes use of a significant upregulation of a member of this pathway, i.e. Wnt4 in fibroids with MED12 mutation compared to those with HMGA2 rearrangements as well as to normal myometrium. Wnt4 is known to be expressed in the mesenchyme of the Müllerian duct, giving rise to the likely tissue of origin of uterine leiomyomas (for review see [27]). The overexpression of Wnt4 in the group of fibroids with mutations of MED12 compared to tumors with HMGA2 rearrangement, as revealed in the experiments underlying the present invention, identifies Wnt4 as a possibly relevant downstream effector of the mutated Med12. Since it has been shown for several cell types that estrogen rapidly induces the expression of Wnt4 in both an estrogen receptor (ER)-dependent and -independent manner [41,42] it is reasonable to assume that the mutated Med12 and estrogen may cooperate in activating their direct transcriptional target Wnt4.

Another question relates to the occurrence of the “fibroid-type mutations” in other groups of benign tumors. Certainly, interesting candidate entities of benign tumors are those sharing with the uterine fibroids recurrent rearrangements of HMGA genes. Quite a number of these entities exist that are not restricted to female genital tumors [8]. For example, HMGA gene rearrangements have been found as frequent abnormalities in lipomas [43], i.e. benign adipose tissue tumors. While in the experiments underlying the present invention no evidence for these mutations in lipomas was obtained, one endometrial polyp was found to be positive which was the only polyp investigated belonging to the rare adenomatous subtype. Rearrangements of HMGA2 due to chromosomal translocations or inversions are a frequent finding in endometrial polyps as well [16-18,44] and suggest that albeit at different frequencies, mechanistically the same two alternate pathways of tumor development exist.

In summary, the results of experiments underlying the present invention provide novel therapeutic targets and molecular markers in the field of gynecological tumors.

### REFERENCES

[1] Baird D D, Dunson D B, Hill M C, Cousins D, Schectman J M: High cumulative incidence of uterine leiomyoma in black and white women: ultrasound evidence. Am J Obstet Gynecol 2003, 188:100-107.
[2] Selo-Ojeme D, Lawal O, Shah J, Mandal R, Pathak S, Selo-Ojeme U, Samuel D: The incidence of uterine leiomyoma and other pelvic ultrasonographic findings in 2,034 consecutive women in a north London hospital. J Obstet Gynaecol 2008, 28:421-423.
[3] Mashal R D, Fejzo M L, Friedman A J, Mitchner N, Nowak R A, Rein M S, Morton C C, Sklar J: Analysis of androgen receptor DNA reveals the independent clonal origins of uterine leiomyomata and the secondary nature of cytogenetic aberrations in the development of leiomyomata. Genes Chromosomes Cancer 1994, 11:1-6.
[4] Quade B J, McLachlin C M, Soto-Wright V, Zuckerman J, Mutter G L, Morton C C: Disseminated peritoneal leiomyomatosis. Clonality analysis by X chromosome inactivation and cytogenetics of a clinically benign smooth muscle proliferation. Am J Pathol 1997, 150:2153-2166.
[5] Canevari R A, Pontes A, Rosa F E, Rainho C A, Rogatto S R: Independent clonal origin of multiple uterine leiomyomas that was determined by X chromosome inactivation and microsatellite analysis. Am J Obstet Gynecol 2005, 193:1395-1403.
[6] Zhang P, Zhang C, Hao J, Sung C J, Quddus M R, Steinhoff M M, Lawrence W D: Use of X-chromosome inactivation pattern to determine the clonal origins of uterine leiomyoma and leiomyosarcoma. Hum Pathol 2006, 37:1350-1356.
[7] Ashar H R, Fejzo M S, Tkachenko A, Zhou X, Fletcher J A, Weremowicz S, Morton C C, Chada K: Disruption of the architectural factor HMGI-C: DNA-binding AT hook motifs fused in lipomas to distinct transcriptional regulatory domains. Cell 1995, 82:57-65.
[8] Schoenmakers E F, Wanschura S, Mols R, Bullerdiek J, Van den Berghe H, Van de Ven W J: Recurrent rearrangements in the high mobility group protein gene, HMGI-C, in benign mesenchymal tumours. Nat Genet 1995, 10:436-444.
[9] Kazmierczak B, Dal Cin P, Wanschura S, Borrmann L, Fusco A, Van den Berghe H, Bullerdiek J: HMGIY is the target of 6p21.3 rearrangements in various benign mesenchymal tumors. Genes Chromosomes Cancer 1998, 23:279-285.
[10] Markowski D N, von Ahsen I, Nezhad M H, Wosniok W, Helmke B M, Bullerdiek J: HMGA2 and the p19Arf-TP53-CDKN1A axis: a delicate balance in the growth of uterine leiomyomas. Genes Chromosomes Cancer 2010, 49:661-668.
[11] Mäkinen N, Mehine M, Tolvanen J, Kaasinen E, Li Y, Lehtonen H J, Gentile M, Yan J, Enge M, Taipale M, Aavikko M, Katainen R, Virolainen E, Böhling T, Koski T A, Launonen V, Sjöberg J, Taipale J, Vahteristo P, Aaltonen L A: MED12, the Mediator Complex Subunit 12 Gene, Is Mutated at High Frequency in Uterine Leiomyomas. Science. 2011 Aug. 25.
[12] Taatjes D J: The human Mediator complex: a versatile, genome-wide regulator of transcription. Trends Biochem Sci 2010, 35:315-322.
[13] Risheg H, Graham J M Jr, Clark R D, Rogers R C, Opitz J M, Moeschler J B, Peiffer A P, May M, Joseph S M, Jones J R, Stevenson R E, Schwartz C E, Friez M J: A recurrent mutation in MED12 leading to R961W causes Opitz-Kaveggia syndrome. Nat Genet 2007, 39:451-453.
[14] Schwartz C E, Tarpey P S, Lubs H A, Verloes A, May M M, Risheg H, Friez M J, Futreal P A, Edkins S, Teague J, Briault S, Skinner C, Bauer-Carlin A, Simensen R J, Joseph S M, Jones J R, Gecz J, Stratton M R, Raymond F L, Stevenson R E: The original Lujan syndrome family has a novel missense mutation (p.N1007S) in the MED12 gene. J Med Genet 2007, 44:472-477.
[15] Sandberg A A: Updates on the cytogenetics and molecular genetics of bone and soft tissue tumors: leiomyoma. Cancer Genet Cytogenet 2005, 158:1-26.
[16] Fletcher J A, Pinkus J L, Lage J M, Morton C C, Pinkus G S: Clonal 6p21 rearrangement is restricted to the mesenchymal component of an endometrial polyp. Genes Chromosomes Cancer 1992, 5:260-263.
[17] Vanni R, Dal Cin P, Marras S, Moerman P, Andria M, Valdes E, Deprest J, Van den Berghe H: Endometrial polyp: another benign tumor characterized by 12q13-q15 changes. Cancer Genet Cytogenet 1993, 68:32-33.
[18] Dal Cin P, Vanni R, Marras S, Moerman P, Kools P, Andria M, Valdes E, Deprest J, Van de Ven W, Van den Berghe H: Four cytogenetic subgroups can be identified in endometrial polyps. Cancer Res 1995, 55:1565-1568.
[19] Bartuma H, Hallor K H, Panagopoulos I, Collin A, Rydholm A, Gustafson P, Bauer H C, Brosjö O, Domanski H A, Mandahl N, Mertens F: Assessment of the clinical and molecular impact of different cytogenetic subgroups in a series of 272 lipomas with abnormal karyotype. Genes Chromosomes Cancer 2007, 46:594-606.
[20] Klemke M, Meyer A, Nezhad M H, Bartnitzke S, Drieschner N, Frantzen C, Schmidt E H, Belge G, Bullerdiek J: Overexpression of HMGA2 in uterine leiomyomas points to its general role for the pathogenesis of the disease. Genes Chromosomes Cancer 2009, 48:171-178.
[21] Robinson, J. P. and International Society for Analytical Cytology: Current protocols in cytometry. 1997, New York: Wiley. Losebl.-Ausg.
[22] Shaffer L G, Slovak M L, Cambel L J, editors: ISCN: An International System for Human Cytogenetic Nomenclature. Basel: S. Karger, 2009.
[23] Dunnen J T and Antonarakis S E: Mutation nomenclature extensions and suggestions to describe complex mutations: a discussion. Hum Mutat 2000, 15:7-12.
[24] Nezhad M H, Drieschner N, Helms S, Meyer A, Tadayyon M, Klemke M, Belge G, Bartnitzke S, Burchardt K, Frantzen C, Schmidt E H, Bullerdiek J: 6p21 rearrangements in uterine leiomyomas targeting HMGA1. Cancer Genet Cytogenet 2010, 203:247-252.
[25] Rein M S, Powell W L, Walters F C, Weremowicz S, Cantor R M, Barbieri R L, Morton C C: Cytogenetic abnormalities in uterine myomas are associated with myoma size. Mol Hum Reprod 1998, 4:83-86.
[26] Hennig Y, Deichert U, Bonk U, Thode B, Bartnitzke S, Bullerdiek J: Chromosomal translocations affecting 12q14˜15 but not deletions of the long arm of chromosome 7 associated with a growth advantage of uterine smooth muscle cells. Mol Hum Reprod 1999, 5:1150-1154.
[27] Deutscher E, Hung-Chang Yao H: Essential roles of mesenchyme-derived beta-catenin in mouse Müllerian duct morphogenesis. Dev Biol 2007, 307:227-236.
[28] Schwartz S M, Marshall L M, Baird D D: Epidemiologic contributions to understanding the etiology of uterine leiomyomata. Environ Health Perspect 2000, 108 Suppl 5:821-827.
[29] Laughlin S K, Schroeder J C, Baird D D: New directions in the epidemiology of uterine fibroids. Semin Reprod Med 2010, 28:204-217.
[30] Baird D D: Invited commentary: uterine leiomyomata-we know so little but could learn so much. Am J Epidemiol 2004, 159:124-126.
[31] Nilbert M, Heim S, Mandahl N, Flodérus U M, Willén H, Mitelman F: Karyotypic rearrangements in 20 uterine leiomyomas. Cytogenet Cell Genet 1988, 49:300-304.
[32] Nilbert M, Heim S, Mandahl N, Flodérus U M, Willén H, Mitelman F: Characteristic chromosome abnormalities, including rearrangements of 6p, del(7q), +12, and t(12;14), in 44 uterine leiomyomas. Hum Genet 1990, 85:605-611.
[33] Meadows K L, Andrews D M, Xu Z, Carswell G K, Laughlin S K, Baird D D, Taylor J A: Genome-wide analysis of loss of heterozygosity and copy number amplification in uterine leiomyomas using the 100K single nucleotide polymorphism array. Exp Mol Pathol 2011, 91:434-439.
[34] Xing Y P, Powell W L, Morton C C: The del(7q) subgroup in uterine leiomyomata: genetic and biologic characteristics. Further evidence for the secondary nature of cytogenetic abnormalities in the pathobiology of uterine leiomyomata. Cancer Genet Cytogenet 1997, 98:69-74.
[35] Rocha P P, Scholze M, Bleiss W, Schrewe H: Med12 is essential for early mouse development and for canonical Wnt and Wnt/PCP signaling. Development 2010, 137:2723-2731.
[36] Tutter A V, Kowalski M P, Baltus G A, Iourgenko V, Labow M, Li E, Kadam S: Role for Med12 in regulation of Nanog and Nanog target genes. J Biol Chem 2009, 284:3709-3718.
[37] Donner A J, Szostek S, Hoover J M, Espinosa J M: CDK8 is a stimulus-specific positive coregulator of p53 target genes. Mol Cell 2007, 27:121-133.
[38] Markowski D N, Winter N, Meyer F, von Ahsen I, Wenk H, Nolte I, Bullerdiek J: p14Arf acts as an antagonist of HMGA2 in senescence of mesenchymal stem cells-implications for benign tumorigenesis. Genes Chromosomes Cancer 2011, 50:489-498.
[39] Mikels A J, Nusse R: Wnts as ligands: processing, secretion and reception. Oncogene 2006, 25:7461-7468.
[40] Tanwar P S, Lee H J, Zhang L, Zukerberg L R, Taketo M M, Rueda B R, Teixeira J M: Constitutive activation of Beta-catenin in uterine stroma and smooth muscle leads to the development of mesenchymal tumors in mice. Biol Reprod 2009, 81:545-552.
[41] Miyakoshi T, Kajiya H, Miyajima K, Takei M, Tobita M, Takekoshi S, Osamura R Y: The expression of Wnt4 is regulated by estrogen via an estrogen receptor alpha-dependent pathway in rat pituitary growth hormone-producing cells. Acta Histochem Cytochem 2009, 2:205-213.
[42] Hou X, Tan Y, Li M, Dey S K, Das S K: Canonical Wnt signaling is critical to estrogen-mediated uterine growth. Mol Endocrinol 2004, 18:3035-3049.
[43] Mandahl, N. and Mertens, F., 2009: Soft tissue tumors. In: Heim, F. and Mitelman, F. (eds.), Cancer Genetics, Wiley, Hoboken, 2009
[44] Dal Cin P, Timmerman D, Van den Berghe I, Wanschura S, Kazmierczak B, Vergote I, Deprest J, Neven P, Moerman P, Bullerdiek J, Van den Berghe H: Genomic changes in endometrial polyps associated with tamoxifen show no evidence for its action as an external carcinogen. Cancer Res 1998, 58:2278-2281.
[45] Osada M, Park H L, Nagakawa Y, Begum S, Yamashita K, Wu G, Kim M S, Trink B, Sidransky D. A novel response element confers p63- and p73-specific activation of the WNT4 promoter. Biochem Biophys Res Commun 2006; 339: 1120-8
[46] Sim E U, Smith A, Szilagi E, Rae F, Ioannou P, Lindsay M H, Little M H. Wnt-4 regulation by the Wilms' tumour suppressor gene, WT1. Oncogene. 2002; 21: 2948-2960.
[47] Devgan V, Mammucari C, Millar S E, Brisken C, Dotto G P. p21WAF1/Cip1 is a negative transcriptional regulator of Wnt4 expression downstream of Notch1 activation. Genes Dev 2005; 19: 1485-1495.
[48] Miyakoshi T, Takei M, Kajiya H, Egashira N, Takekoshi S, Teramoto A, Osamura R Y. Expression of Wnt4 in human pituitary adenomas regulates activation of the beta-catenin-independent pathway. Endocr Pathol. 2008 Winter; 19:261-73.
[49] Xiaoyang Wan, Wei Ji, Xue Mei, Jiangang Zhou, Jing-xia Liu, Chengchi Fang, Wuhan Xiao. Negative Feedback Regulation of Wnt4 Signaling by EAF1 and EAF2/U19. PLoS One. 2010; 5: e9118.
[50] Qiu W, Wang X, Leibowitz B, Liu H, Barker N, Okada H, Oue N, Yasui W, Clevers H, Schoen R E, Yu J, Zhang L. Chemoprevention by nonsteroidal anti-inflammatory drugs eliminates oncogenic intestinal stem cells via SMAC-dependent apoptosis. Proc Natl Acad Sci USA. 2010; 107: 20027-32.
[51] Tutter A V, Fryer C J, Jones K A. Chromatin-specific regulation of LEF-1-beta-catenin transcription activation and inhibition in vitro. Genes Dev. 2001; 15:3342-54.
[52] Boon E M, Keller J J, Wormhoudt T A, Giardiello F M, Offerhaus G J, van der Neut R, Pals S T. Sulindac targets nuclear beta-catenin accumulation and Wnt signalling in adenomas of patients with familial adenomatous polyposis and in human colorectal cancer cell lines. Br. J. Cancer. 2004; 90:224-9.
[53] Chen B, Dodge M E, Tang W, Lu J, Ma Z, Fan C W, Wei S, Hao W, Kilgore J, Williams N S, Roth M G, Amatruda J F, Chen C, Lum L. Small molecule-mediated disruption of Wnt-dependent signaling in tissue regeneration and cancer. Nat Chem Biol. 2009; 5:100-7.
[54] Giancotti, V. et al. Elevated levels of a specific class of nuclear phosphoproteins in cells transformed with ras and v-mos oncogenes and by co-transfection with c-myc and polyoma middle T genes. EMBO J. 6, 1981-1987 (1987)
[55] Patel, U. A. et al. Expression and eDNA cloning of human HMGI-C phosphoprotein. Biochem. Biophys. Res. Commun 201, 63-70 (1994)
[56] Manfioletti, G. et al. eDNA cloning of the HMGI-C phosphoprotein, a nuclear protein associated with neoplastic and undifferentiated phenotypes. Nucl. Acids Res. 19, 6793-6797 (1991).
[57] Fang G, Bhardwaj N, Robilotto R, Gerstein M B., Getting started in gene orthology and functional analysis. PLoS Comput Biol. 2010 Mar. 26; 6(3):e1000703.

TABLE 1
Summary of the clinical and cytogenetic findings as well as of MED12 mutations in a total of 80 uterine fibroids investigated.
In some cases chromosomal rearrangements involving the long arm of chromosome 12 without involvement of the 12q14~15 segment has
been found by conventional cytogenetics but FISH (Fluorescent In Situ Hybridization) using an appropriate break-apart probe detected
rearrangements of the HMGA2 locus.

| | | | clonal | | |
|---|---|---|---|---|---|
| | age | size¹⁾ | chromosome | | MED12 |
| no. | [y] | [cm] | aberrations | karyotype²⁾ | mutation |
| 503.1 | 40 | 4.0 | 12q14~15 rearr. | 46, XX, inv(5)(q15q31~33), t(12; 14)(q15; q24)[13] | no |
| 515.1 | 46 | 3.0 | 12q14~15 rearr. | no conventional karyotype; HMGA2 rearrangement detected only by FISH | no |
| | | | FISH | | |
| 520.1 | 38 | 5.0 | normal | 46, XX[7] | c.130G > C |
| 523.1 | 33 | n.d. | 12q14~15 rearr. | 45, XX, t(12; 14)(q15; q24), der(14)t(12; 14)(q15; q24), −22[8] | no |
| 533.1 | 41 | 6.0 | 12q14~15 rearr. | 46, XX, r(1), t(1; 12; 14)(p36.3; q14; q24)[19] | no |
| 536.3 | 46 | 3.0 | del(7)(q) | 46, XX, del(7)(q21.2q31.2)[6] | no |
| 538.4 | 36 | 3.0 | normal | 46, XX[6] | c.131G > C |
| 538.7 | 36 | 9.0 | normal | 46, XX[6] | c.131G > A |
| 540.1 | 49 | 4.0 | normal | 46, XX[10] | c.130G > T |
| 540.2 | 49 | n.r. | normal | 46, XX[4] | c.131G > A |
| 540.3 | 49 | n.r. | normal | 46, XX[10] | c.131G > A |
| 541.1 | 37 | 7.0 | 12q14~15 rearr. | 46, XX, t(12; 14)(q15; q24)[5]/46, XX[9] | no |
| 544.2 | 49 | 4.0 | del(7)(q) | 46, XX, del(7)(q22q32)[2]/46, XX[4] | c.123-134del(12) |
| 545.1 | 47 | 5.0 | 12q14~15 rearr. | 46, XX, t(12; 14)(q15; q24)[9]/46, XX[3] | no |
| 552.1 | 49 | 3.0 | normal | 46, XX[12] | no |
| 552.2 | 49 | 10.0 | 12q14~15 rearr. | 46, XX, t(2; 12)(q33; q13)[17] | no |
| 556.1 | 42 | 5.0 | 12q14~15 rearr. | 46, XX, t(3; 5; 12)(q23~25; p13~15; q13~15)[11]/45, XX, idem, −22[10] | no |
| 557.1 | 38 | 1.0 | normal | 46, XX[10] | c.130 G > C |
| 557.2 | 38 | 3.0 | normal | 46, XX[10] | c.130 G > A |
| | | | | | c.131 G > A |
| 557.3 | 38 | 2.0 | normal | 46, XX[10] | c.131G > T |
| 557.4 | 38 | 4.0 | normal | 46, XX[13] | no |
| 558.1 | 34 | 1.0 | normal | 46, XX[13] | c.130 G > A |
| 565.1 | 42 | 10.0 | normal | 46, XX[14] | no |
| 579.1 | 49 | 1.5 | 12q14~15 rearr. | 46, XX, t(12; 15; 14)(q15; q26; q24)[20] | no |
| 580.1 | 40 | 8.0 | 12q14~15 rearr. | 46, XX, der(7)del(7)(p)del(7)(q), add(8)(q), add(10)(q), t(12; 14)(q15; q24)[19] | no |
| | | | plus del(7)(q) | | |
| 583.1 | 40 | 5.5 | normal | 46, XX[16] | c.130 G > A |
| 610.1 | 53 | 6.0 | normal | 46, XX[10] | c.131 G > A |
| 610.2 | 53 | 4.5 | normal | 46, XX[13] | c.131 G > A |
| 610.3 | 53 | 3.5 | 6p21~23 rearr. | 46, XX, t(6; 10)(p23; q23)[5]/46, XX[7] | c.130 G > T |
| 610.4 | 53 | 3.5 | normal | 46, XX[14] | c.131 G > T |
| 610.5 | 53 | 3.5 | normal | 46, XX[14] | c.130 G > C |
| 610.6 | 53 | 3.0 | normal | 46, XX[17] | c.130 G > C |
| 612.1 | 44 | 6.0 | 12q14~15 rearr. | 46, XX, t(12; 14)(q15; q24)[13]/46, XX, der(l)r(1; ?), t(12; 14)(q15; q24)[4] | no |
| 613.1 | 39 | 11.0 | normal | 46, XX[15] | c.130 G > A |
| 613.2 | 39 | 7.0 | normal | 46, XX[15] | c.131 G > A |
| 613.3 | 39 | 7.0 | normal | 46, XX[16] | c.130 G > A |
| 613.4 | 39 | 4.5 | 6p21~23 rearr. | 46, XX, t(6; 11)(p23; q21)[4]/46, XX[12] | 130 G > C |
| 613.5 | 39 | 8.0 | normal | 46, XX[9] | no |
| 614.1 | 56 | 2.0 | del(7)(q) | 46, XX, del(7)(q22q32)[2]/46, XX[21] | c.131 G > T |
| 614.2 | 56 | 1.5 | normal | 46, XX[17] | no. |
| 615.2 | 47 | 3.0 | normal | 46, XX[14] | no |
| 617.1 | 44 | 8.0 | 12q14~15 rearr. | 46, XX, der(l)del(1)(p22), der(3)?t(1; 3)(p22; q?), der(5)del(5), der(12)t(12; ?)(q2 | no |
| | | | | 4.3; ?), −14, −20, +mar1, +mar2[6] | |
| 619.1 | 46 | 3.0 | normal | 46, XX[14] | c.131 G > A |
| 619.2 | 46 | 8.0 | normal | 46, XX[15] | c.130 G > A |
| 621.1 | 42 | 2.5 | 6p21~23 rearr. | 46, XX, t(6; 11)(p21; p15)[7]/46, XX[14] | c.130 G > A |
| 621.2 | 42 | 2.0 | normal | 46, XX[12] | c.131 G > A |
| 628.2 | 57 | 1.5 | 12q14~15 rearr. | 46, XX, ?ins(12; 14)(q15; q31q24)[5]/46, XX[14] | no |
| 632.1 | 47 | 4.0 | 12q14~15 rearr. | 46, XX, t(12; 14)(q15; q24)[12]/46, XX, del(4)(q31or | no |
| | | | | q32), der(10), ?t(10; 14)(q24; q32), t(12; 14)(q15; q24)[9]/45, XX, der(1), ?t(1; 2), | |
| | | | | −2, add(7)(?q36), t(12; 14)(q15; q24)[2] | |
| 635.1 | 48 | n.r. | 12q14~15 rearr. | 46, XX, der(10), del(12)(q13 or q14)[18] | no |
| 640.2 | 60 | 2.0 | normal | 46, XX[15] | c.131 G > A |
| 642.3 | 63 | 6.0 | normal | 46, XX[8] | c.130 G > C |
| 643.1 | 52 | 1.0 | normal | 46, XX[7] | c.128 A > C |
| 643.2 | 52 | 6.0 | 12q14~15 rearr. | 46, XX, t(12; 14)(q15; q24)[14] | no |
| 645.1 | 56 | 8.0 | 12q14~15 rearr. | 45, XX, r(1), der(13; 14)(q10; q10)t(12; 14)(q15; q24)[20]/44, XX, | no |
| | | | | −1, der(13; 14)(q10; q10)t(12; 14)(q15; q24)[6] | |
| 646.1 | 47 | 9.5 | 12q14~15 rearr. | 46, XX, t(2; 12)(p21; p13)[11] | no |
| | | | | FISH detected a HMGA2 rearrangement | |
| 649.1 | 42 | 2.0 | normal | 46, XX[14] | no |
| 653.1 | 50 | 1.0 | normal | 46, XX[14] | c.131 G > A |
| 654.1 | 43 | 3.0 | normal | 46, XX[8] | c.131 G > A |
| 658.1 | 47 | 3.0 | 6p21~23 rearr. | 46, XX, t(6; 10)(p21; q22)[13]/46, XX[8] | no |
| 658.2 | 47 | 3.0 | normal | 46, XX[13] | c.130 G > C |
| 668.1 | 46 | 3.0 | normal | 46, XX[10] | c.130 G > C |
| 668.2 | 46 | 2.0 | normal | 46, XX[11] | c.130 G > A |
| 668.3 | 46 | 2.5 | normal | 46, XX[7] | no |
| 675.2 | 64 | 8.0 | 12q14~15 rearr. | 46, XX, t(12; 14)(q15; q24)[21] | no |
| 677.2 | 54 | 2.5 | normal | 46, XX[7] | c.131 G > A |
| 677.3 | 54 | 8.0 | 12q14~15 rearr. | 46, XX, add(1)(p13), r(1)?(p36.3q25), add(7)(q22), der(10)t(1; 10)(q25q22), der | no |
| | | | | (12)add(12)(p11.2)add(q12), add(13)(q12)[17]/46, XX[3] | |
| 677.4 | 54 | 3.0 | normal | 46, XX[30] | c.131 G > A |
| 682.2 | 69 | 1.0 | normal | 46, XX[38] | c.131 G > A |
| 685.1 | 52 | 1.0 | normal | 46, XX[17] | c.131 G > T |
| 685.2 | 52 | 5.5 | rearr. of | 46, XX, der(7)add(7)(p)add(7)(q)[8] | c.131 G > A |
| | | | chromosome 7 | | |
| 688.1 | 60 | 1.0 | normal | 46, XX[21] | no |
| 689.3 | 47 | 2.5 | normal | 46, XX[16] | c.131 G > T |
| 689.4 | 47 | 8.0 | normal | 46, XX[10] | c.131 G > A |
| 696.2 | 49 | 4.0 | 6p21~23 rearr. | 46, XX, del(1)(q42), t(4; 6; 14)?(q32; p21.3; q24)[11] | no |

¹⁾largest diameter,
²⁾according to ISCN [43], but the number of metaphases in square brackets refers to those analyzed from the primary culture only. (rearr. = rearrangement)

TABLE 2
DNA-Sequences
Med12 - mRNA according to Genebank entry No.: NM_005120.2.
cDNA corresponding to bases 200 to 6730 of the mRNA-sequence
is underlined; Nucleotides c.127 to c.132 encoding codons
43 to 44 of the MED12-gene are marked in bold and italic

ATTGTCCGATGGTTCCCGGCGTACCTCGGCTTCCCTCGGTAGTTTCCGGCAATGGTCGAGAGTTTCTA
ACGTGCCCCCTTGTTGTCTCTCGGCCGCCGTCCTCTCAACCACCGCCCCCCTTTTCGGCTCCCTCTCC
CCCTTCCCGTTCCCCCAGTCAGCCTGGCCCTGCTGGTGCCTCCGGCGCTACGGGCTGGGCAAGATGGC
GGCCTTCGGGATCTTGAGCTACGAACACCGGCCCCTGAAGCGGCCGCGGCTGGGGCCTCCCGATGTTT
ACCCTCAGGACCCCAAACAGAAGGAGGATGAACTGACGGCCTTGAATGTAAAA [symbol] TTCAATAAC
CAGCCTGCTGTCTCTGGGGATGAGCATGGCAGTGCCAAGAACGTCAGCTTCAATCCTGCCAAGATCAG
TTCCAACTTCAGCAGCATTATTGCAGAGAAATTACGTTGTAATACCCTTCCTGACACTGGTCGCAGGA
AGCCCCAAGTGAACCAGAAGGATAACTTCTGGCTGGTGACTGCACGATCCCAGAGTGCCATTAACACT
TGGTTCACTGACTTGGCTGGCACCAAGCCACTCACGCAACTAGCCAAAAAGGTCCCCATTTTCAGTAA
GAAGGAAGAGGTGTTTGGGTACTTAGCCAAATACACAGTGCCTGTGATGCGGGCTGCCTGGCTCATTA
AGATGACCTGTGCCTACTATGCAGCAATCTCTGAGACCAAGGTTAAGAAGAGACATGTTGACCCTTTC
ATGGAATGGACTCAGATCATCACCAAGTACTTATGGGAGCAGTTACAGAAGATGGCTGAATACTACCG
GCCAGGGCCTGCAGGAAGTGGGGGCTGTGGTTCCACGATAGGGCCCTTGCCCCATGATGTAGAGGTGG
CAATCCGGCAGTGGGATTACACCGAGAAGCTGGCCATGTTCATGTTTCAGGATGGAATGCTGGACAGA
CATGAGTTCCTGACCTGGGTGCTTGAGTGTTTTGAGAAGATCCGCCCTGGAGAGGATGAATTGCTTAA
ACTGCTGCTGCCTCTGCTTCTCCGATACTCTGGGGAATTTGTTCAGTCTGCATACCTGTCCCGCCGGC
TTGCCTACTTCTGTACACGGAGACTGGCCCTGCAGCTGGATGGTGTGAGCAGTCACTCATCTCATGTT
ATATCTGCTCAGTCAACAAGCACGCTACCCACCACCCCTGCTCCTCAGCCCCCAACTAGCAGCACACC
CTCGACTCCCTTTAGTGACCTGCTTATGTGCCCTCAGCACCGGCCCCTGGTTTTTGGCCTCAGCTGTA
TCCTACAGACCATCCTCCTGTGCTGTCCTAGTGCCTTGGTTTGGCACTACTCACTGACTGATAGCAGA
ATTAAGACCGGCTCACCACTTGACCACTTGCCTATTGCCCCGTCCAACCTGCCCATGCCAGAGGGTAA
CAGTGCCTTCACTCAGCAGGTCCGTGCAAAGTTGCGGGAGATCGAGCAGCAGATCAAGGAGCGGGGAC
AGGCAGTTGAAGTTCGCTGGTCTTTCGATAAATGCCAGGAAGCTACTGCAGGCTTCACCATTGGACGG
GTACTTCATACTTTGGAAGTGCTGGACAGCCATAGTTTTGAACGCTCTGACTTCAGCAACTCTCTTGA
CTCCCTTTGTAACCGAATCTTTGGATTGGGACCTAGCAAGGATGGGCATGAGATCTCCTCAGATGATG
ATGCTGTGGTGTCATTGCTATGTGAATGGGCTGTCAGCTGCAAGCGTTCTGGTCGGCATCGTGCTATG
GTGGTAGCCAAGCTCCTGGAGAAGAGACAGGCGGAGATTGAGGCTGAGCGTTGTGGAGAATCAGAAGC
CGCAGATGAGAAGGGTTCCATCGCCTCTGGCTCCCTTTCTGCTCCCAGTGCTCCCATTTTCCAGGATG
TCCTCCTGCAGTTTCTGGATACACAGGCTCCCATGCTGACGGACCCTCGAAGTGAGAGTGAGCGGGTG
GAATTCTTTAACTTAGTACTGCTGTTCTGTGAACTGATTCGACATGATGTTTTCTCCCACAACATGTA
TACTTGCACTCTCATCTCCCGAGGGGACCTTGCCTTTGGAGCCCCTGGTCCCCGGCCTCCCTCTCCCT
TTGATGATCCTGCCGATGACCCAGAGCACAAGGAGGCTGAAGGCAGCAGCAGCAGCAAGCTGGAAGAT
CCAGGGCTCTCAGAATCTATGGACATTGACCCTAGTTCCAGTGTTCTCTTTGAGGACATGGAGAAGCC
TGATTTCTCATTGTTCTCCCCTACTATGCCCTGTGAGGGGAAGGGCAGTCCATCCCCTGAGAAGCCAG
ATGTCGAGAAGGAGGTGAAGCCCCCACCCAAGGAGAAGATTGAAGGGACCCTTGGGGTTCTTTACGAC
CAGCCACGACACGTGCAGTACGCCACCCATTTTCCCATCCCCCAGGAGGAGTCATGCAGCCATGAGTG
CAACCAGCGGTTGGTCGTACTGTTTGGGGTGGGAAAGCAGCGAGATGATGCCCGCCATGCCATCAAGA
AAATCACCAAGGATATCTTGAAGGTTCTGAACCGCAAAGGGACAGCAGAAACTGACCAGCTTGCTCCT
ATTGTGCCTCTGAATCCTGGAGACCTGACATTCTTAGGTGGGGAGGATGGGCAGAAGCGGCGACGCAA
CCGGCCTGAAGCCTTCCCCACTGCTGAAGATATCTTTGCTAAGTTCCAGCACCTTTCACATTATGACC
AACACCAGGTCACGGCTCAGGTCTCCCGGAATGTTCTGGAGCAGATCACGAGCTTTGCCCTTGGCATG
TCATACCACTTGCCTCTGGTGCAGCATGTGCAGTTCATCTTCGACCTCATGGAATATTCACTCAGCAT
CAGTGGCCTCATCGACTTTGCCATTCAGCTGCTGAATGAACTGAGTGTAGTTGAGGCTGAGCTGCTTC
TCAAATCCTCGGATCTGGTGGGCAGCTACACTACTAGCCTGTGCCTGTGCATCGTGGCTGTCCTGCGG
CACTATCATGCCTGCCTCATCCTCAACCAGGACCAGATGGCACAGGTCTTTGAGGGGCTGTGTGGCGT
CGTGAAGCATGGGATGAACCGGTCCGATGGCTCCTCTGCAGAGCGCTGTATCCTTGCTTATCTCTATG
ATCTGTACACCTCCTGTAGCCATTTAAAGAACAAATTTGGGGAGCTCTTCAGCGACTTTTGCTCAAAG
GTGAAGAACACCATCTACTGCAACGTGGAGCCATCGGAATCAAATATGCGCTGGGCACCTGAGTTCAT
GATCGACACTCTAGAGAACCCTGCAGCTCACACCTTCACCTACACGGGGCTAGGCAAGAGTCTTAGTG
AGAACCCTGCTAACCGCTACAGCTTTGTCTGCAATGCCCTTATGCACGTCTGTGTGGGGCACCATGAT
CCCGATAGGGTGAATGACATCGCAATCCTGTGTGCAGAGCTGACCGGCTATTGCAAGTCACTGAGTGC
AGAATGGCTAGGAGTGCTTAAGGCCTTGTGCTGCTCCTCTAACAATGGCACTTGTGGTTTCAACGATC
TCCTCTGCAATGTTGATGTCAGTGACCTATCTTTTCATGACTCGCTGGCTACTTTTGTTGCCATCCTC
ATCGCTCGGCAGTGTTTGCTCCTGGAAGATCTGATTCGCTGTGCTGCCATCCCTTCACTCCTTAATGC
TGCTTGTAGTGAACAGGACTCTGAGCCAGGGGCCCGGCTTACCTGCCGCATCCTCCTTCACCTTTTCA
AGACACCGCAGCTCAATCCTTGCCAGTCTGATGGAAACAAGCCTACAGTAGGAATCCGCTCCTCCTGC
GACCGCCACCTGCTGGCTGCCTCCCAGAACCGCATCGTGGATGGAGCCGTGTTTGCTGTTCTCAAGGC
TGTGTTTGTACTTGGGGATGCGGAACTGAAAGGTTCAGGCTTCACTGTGACAGGAGGAACAGAAGAAC
TTCCAGAGGAGGAGGGAGGAGGTGGCAGTGGTGGTCGGAGGCAGGGTGGCCGCAACATCTCTGTGGAG
ACAGCCAGTCTGGATGTCTATGCCAAGTACGTGCTGCGCAGCATCTGCCAACAGGAATGGGTAGGAGA
ACGTTGCCTTAAGTCTCTGTGTGAGGACAGCAATGACCTGCAAGACCCAGTGTTGAGTAGTGCCCAGG
CGCAGCGCCTCATGCAGCTCATTTGCTATCCACATCGACTGCTGGACAATGAGGATGGGGAAAACCCC
CAGCGGCAGCGCATAAAGCGCATTCTCCAGAACTTGGACCAGTGGACCATGCGCCAGTCTTCCTTGGA
GCTGCAGCTCATGATCAAGCAGACCCCTAACAATGAGATGAACTCCCTCTTGGAGAACATCGCCAAGG
CCACAATCGAGGTTTTCCAACAGTCAGCAGAGACAGGGTCATCTTCTGGAAGTACTGCAAGCAACATG
CCCAGCAGCAGCAAGACCAAGCCTGTGCTCAGCTCTCTAGAGCGCTCTGGTGTATGGCTGGTGGCCCC
CCTCATTGCTAAACTGCCCACCTCAGTCCAGGGACATGTGTTAAAGGCTGCTGGGGAAGAATTGGAGA
AGGGTCAGCACCTGGGTTCCTCTTCACGCAAAGAACGTGATCGACAAAAGCAGAAGAGCATGTCCCTA
TTGAGCCAGCAGCCCTTCTTATCGCTGGTGCTAACATGTCTGAAAGGGCAGGATGAACAACGCGAGGG
ACTCCTTACCTCCCTCTACAGCCAGGTGCACCAGATTGTGAATAATTGGCGAGATGACCAGTACTTAG
ATGATTGCAAACCAAAGCAGCTTATGCATGAGGCACTCAAACTGCGGCTCAACCTGGTGGGGGGCATG
TTTGACACGGTGCAGCGCAGCACCCAGCAGACCACGGAGTGGGCCATGCTCCTCCTGGAGATCATCAT
CAGCGGCACTGTCGACATGCAGTCCAACAATGAGCTCTTCACTACTGTGTTGGACATGCTGAGCGTGC
TCATCAATGGGACATTGGCTGCAGACATGTCTAGCATCTCGCAAGGTAGCATGGAGGAAAACAAGCGT
GCATACATGAACCTGGCGAAGAAGTTGCAGAAGGAGTTGGGGGAGCGCCAGTCAGACAGTCTGGAAAA
GGTTCGCCAGCTGCTGCCACTGCCCAAGCAGACCCGAGATGTCATCACGTGTGAGCCACAGGGCTCCC
TTATCGATACCAAGGGCAACAAGATTGCTGGCTTCGATTCCATCTTCAAGAAGGAGGGTCTACAGGTT
TCCACCAAACAGAAGATCTCGCCCTGGGATCTTTTTGAGGGGTTGAAGCCGTCAGCACCACTCTCTTG
GGGCTGGTTTGGAACAGTCCGAGTGGACCGGCGAGTGGCTCGAGGAGAGGAGCAGCAGCGGTTGCTGC
TCTACCACACACACCTGAGGCCCCGGCCCCGCGCCTATTACCTGGAGCCACTGCCACTGCCCCCAGAA
GATGAGGAGCCGCCTGCTCCTACCCTGCTAGAGCCTGAGAAAAAGGCTCCAGAGCCCCCCAAAACTGA
CAAACCGGGGGCTGCTCCACCCAGTACTGAGGAACGCAAGAAGAAGTCCACCAAGGGCAAGAAACGCA
GCCAGCCAGCTACCAAGACAGAGGACTATGGAATGGGCCCGGGTCGGAGCGGCCCTTATGGTGTGACA
GTGCCTCCGGACCTCCTGCACCACCCAAACCCTGGTTCTATAACACACCTTAACTACAGGCAAGGCTC
CATAGGCCTGTACACCCAGAACCAGCCACTACCTGCAGGTGGCCCTCGTGTGGACCCATACCGTCCTG
TGCGCTTACCAATGCAGAAGCTGCCCACCCGACCAACTTACCCTGGAGTGCTGCCCACAACCATGACT
GGCGTCATGGGTTTAGAACCCTCCTCTTATAAGACCTCTGTGTACCGGCAGCAGCAACCTGCGGTGCC
CCAAGGACAGCGCCTTCGCCAACAGCTCCAGCAGAGTCAGGGCATGTTGGGACAGTCATCTGTCCATC
AGATGACTCCCAGCTCTTCCTACGGTTTGCAGACTTCCCAGGGCTATACTCCTTATGTTTCTCATGTG
GGATTGCAGCAACACACAGGCCCTGCAGGTACCATGGTGCCCCCCAGCTACTCCAGCCAGCCTTACCA
GAGCACCCACCCTTCTACCAATCCTACTCTTGTAGATCCTACCCGCCACCTGCAACAGCGGCCCAGTG
GCTATGTGCACCAGCAGGCCCCCACCTATGGACATGGACTGACCTCCACTCAAAGGTTTTCACACCAG
ACACTGCAGCAGACACCCATGATAAGTACCATGACTCCAATGAGTGCCCAGGGCGTCCAGGCAGGCGT
CCGTTCAACAGCCATCCTACCTGAGCAGCAGCAGCAGCAGCAACAGCAGCAACAGCAACAGCAGCAGC
AGCAGCAACAGCAACAGCAGCAGCAGCAGCAGCAGTACCACATCCGGCAGCAGCAGCAGCAGCAGATC
CTGCGGCAGCAGCAGCAACAGCAACAGCAGCAGCAGCAGCAGCAGCAACAGCAACAGCAGCAGCAGCA
ACAGCAACAACAGCAACACCAGCAGCAACAGCAGCAACAGGCGGCTCCTCCCCAACCCCAGCCCCAGT
CCCAGCCCCAGTTCCAGCGCCAGGGGCTTCAGCAGACCCAGCAGCAGCAACAGACAGCAGCTTTGGTC
CGGCAACTTCAACAACAGCTCTCTAATACCCAGCCACAGCCCAGTACCAACATATTTGGACGCTACTG
AGCCACCTGGAGGAACTGCTTGTGCACTGGATGTGGCCCCACCCTTTCCTCTTAATTCCCAATCCCAT
TCCTGGGCTAGCACCAGTAGTGGTTGGGGCCCTCCCCTCAGGCTCCATTTTTAATAAGTTTTTAGTAT
TTTTGTTAATGTGAGGCATTGAGCTGTTGGGTTTTGTATATTATTTATATAGAGACCCCAGAGCTGTT
GCACCCAATACACAGAGCTTCTTTGCAAAGGGAAAAAAAAAAAAAAAAA SEQ ID NO: 1

1. A method of treating a benign or malignant gynaecological tumor comprising using a Wnt4 inhibitor to disturb or inhibit cell signalling permitted by Wnt4 activity thereby reducing growth and proliferative potential of cells from the gynecological tumor.

2. The method of claim 1, wherein the tumor is selected from the group consisting of endometrial polyps, endometriosis, adenomyosis, leiomyosarcomas of the uterus, aggressive angiomyxomas, endometrial carcinomas and Müllerian mixed tumors.

3. The method of claim 1, wherein the tumor is uterine leiomyoma (UL).

4. The method of claim 1, wherein the Wnt4 inhibitor is selected from the group consisting of small molecules, antibodies, antigen-binding antibody fragments, aptamers, spiegelmers, siRNA and miRNA.

5. A method to determine the response potential of a tumor of claim 1 to a treatment by a Wnt4 inhibitor, comprising:
(a) detecting at least one MED12-mutation affecting the sequence CAAGGT (corresponding to bases 326 to 331 of the MED12-mRNA-sequence of SEQ ID NO: 1) encoding codons 43 to 44 of the MED12-gene, in a test sample derived from a patient, wherein the presence of said at least one MED12-mutation is indicative for a tumor responsive to treatment with Wnt4 inhibitors; and/or
(b) determining Wnt4 expression in a test sample derived from a patient, wherein an enhanced expression compared to a control sample is indicative for a tumor responsive to treatment with Wnt4 inhibitors.

6. A method for detection of at least one MED12-mutation as defined in claim 5 for use in determining the growth potential of a uterine leiomyoma (UL) tumor comprising detecting at least one MED12-mutation in a test sample derived from a patient, wherein c.130 or c.131G>A transitions at codons 43 or 44 of the MED12-gene are indicative of a higher growth potential of the tumor compared to a tumor comprising different MED12-mutations at codons 43 or 44.

7. A method for diagnosing a pituitary tumor, a prostate tumor or a prostate hyperplasia comprising detecting MED12 mutations in a test sample derived from a respective pituitary gland or prostate.

8. A method for differential diagnosis of uterine smooth muscle tumors comprising:
(a) detection of a mutation in the MED12 gene and its expression; and/or
(b) determination of expression of the gene encoding high mobility group protein AT-hook 2 (HMGA2) and/or of rearrangements of the HMGA1 and/or HMGA2 gene locus in a test sample from a patient; wherein:
(i) increased and/or ectopic HMGA2 expression, absence of a MED12 mutation, and absence of rearrangements of the HMGA2 gene locus are indicative for a malignant smooth muscle tumor;
(ii) increased and/or ectopic HMGA2 expression, and presence of rearrangements of the HMGA2 gene locus are indicative for a benign smooth muscle tumor;
(iii) presence of a MED12 mutation, normal HMGA2 and MED12 expression and absence of rearrangements of the HMGA2 gene locus are indicative for a benign smooth muscle tumor;
(iv) presence of rearrangements of the HMGA1 gene locus, normal HMGA2 expression, absence of a MED12 mutation and absence of rearrangements of the HMGA2 gene locus are indicative for a benign smooth muscle tumor;
(v) presence of a MED12 mutation, normal HMGA2 expression, not detectable MED12 expression and absence of rearrangements of the HMGA2 gene locus are indicative for a malignant smooth muscle tumor; and
(vi) increased and/or ectopic HMGA2 expression and presence of a MED12 mutation are indicative for a malignant smooth muscle tumor.

9. The method of claim 8, wherein the MED12 gene is analyzed for presence of a mutation affecting the sequence CAAGGT (corresponding to bases 326 to 331 of the MED12-mRNA-sequence of SEQ ID NO: 1) encoding codons 43 to 44.

10. The method of claim 8, wherein the malignant smooth muscle tumor is leiomyosarcoma and the benign smooth muscle tumor is leiomyoma.

11. A method for identification of suitable mammalian models for different types of smooth muscle tumors comprising the method of claim 8, wherein the presence of a MED12 mutation, HMGA2 expression and/or presence of rearrangements of the HMGA2 and/or HMGA1 gene locus are analyzed in respect of homologues of the human MED12, HMGA1 and HMGA2 genes in a test sample of the respective mammal.

12. A kit useful in a method of claim 5, comprising one or more reagents for detecting MED12 mutations.

13. The kit of claim 12, wherein the reagents comprise an antibody or a nucleic acid.

14. The kit of claim 12, comprising primers for the amplification of a fragment of the genomic template DNA region comprising the MED12 locus, for amplification of a target cDNA-fragment generated from a MED12-mRNA and/or for sequencing of said amplified fragments.

15. The kit of claim 12, comprising primers for the quantification of Wnt4 expression in a test sample.

16. The kit of claim 12, comprising reagents for the quantification of HMGA2 expression, for detection of HMGA2 expression and/or of rearrangements of the HMGA2 and/or HMGA1 gene locus in a test sample.


SEQUENCE LISTING (7 sequences)

SEQ ID NO: 1 - DNA, 6985 bp, Homo sapiens
  CDS (200)..(6730): coding sequence of Med12
attgtccgatggttcccggcgtacctcggcttccctcggtagtttccggcaatggtcgagagtttctaacgtgcccccttgttgtctctcggccgccgtcctctcaaccaccgccccccttttcggctccctctcccccttcccgttcccccagtcagcctggccctgctggtgcctccggcgctacgggctgggcaagatggcggccttcgggatcttgagctacgaacaccggcccctgaagcggccgcggctggggcctcccgatgtttaccctcaggaccccaaacagaaggaggatgaactgacggccttgaatgtaaaacaaggtttcaataaccagcctgctgtctctggggatgagcatggcagtgccaagaacgtcagcttcaatcctgccaagatcagttccaacttcagcagcattattgcagagaaattacgttgtaatacccttcctgacactggtcgcaggaagccccaagtgaaccagaaggataacttctggctggtgactgcacgatcccagagtgccattaacacttggttcactgacttggctggcaccaagccactcacgcaactagccaaaaaggtccccattttcagtaagaaggaagaggtgtttgggtacttagccaaatacacagtgcctgtgatgcgggctgcctggctcattaagatgacctgtgcctactatgcagcaatctctgagaccaaggttaagaagagacatgttgaccctttcatggaatggactcagatcatcaccaagtacttatgggagcagttacagaagatggctgaatactaccggccagggcctgcaggaagtgggggctgtggttccacgatagggcccttgccccatgatgtagaggtggcaatccggcagtgggattacaccgagaagctggccatgttcatgtttcaggatggaatgctggacagacatgagttcctgacctgggtgcttgagtgttttgagaagatccgccctggagaggatgaattgcttaaactgctgctgcctctgcttctccgatactctggggaatttgttcagtctgcatacctgtcccgccggcttgcctacttctgtacacggagactggccctgcagctggatggtgtgagcagtcactcatctcatgttatatctgctcagtcaacaagcacgctacccaccacccctgctcctcagcccccaactagcagcacaccctcgactccctttagtgacctgcttatgtgccctcagcaccggcccctggtttttggcctcagctgtatcctacagaccatcctcctgtgctgtcctagtgccttggtttggcactactcactgactgatagcagaattaagaccggctcaccacttgaccacttgcctattgccccgtccaacctgcccatgccagagggtaacagtgccttcactcagcaggtccgtgcaaagttgcgggagatcgagcagcagatcaaggagcggggacaggcagttgaagttcgctggtctttcgataaatgccaggaagctactgcaggcttcaccattggacgggtacttcatactttggaagtgctggacagccatagttttgaacgctctgacttcagcaactctcttgactccctttgtaaccgaatctttggattgggacctagcaaggatgggcatgagatctcctcagatgatgatgctgtggtgtcattgctatgtgaatgggctgtcagctgcaagcgttctggtcggcatcgtgctatggtggtagccaagctcctggagaagagacaggcggagattgaggctgagcgttgtggagaatcagaagccgcagatgagaagggttccatcgcctctggctccctttctgctcccagtgctcccattttccaggatgtcctcctgcagtttctggatacacaggctcccatgctgacggaccctcgaagtgagagtgagcgggtggaattctttaacttagtactgctgttctgtgaactgattcgacatgatgttttctcccacaacatgtatacttgcactctcatctcccgaggggaccttgcctttggagcccctggtccccggcctccctctccctttgatgatcctgccgatgacccagagcacaaggaggctgaaggcagcagcagcagcaagctggaagatccagggctctcagaatctatggacattgaccctagttccagtgttctctttgaggacatggagaagcctgatttctcattgttctcccctactatgccctgtgaggggaagggcagtccatcccctgagaagccagatgtcgagaaggaggtgaagcccccacccaaggagaagattgaagggacccttggggttctttacgaccagccacgacacgtgcagtacgccacccattttcccatcccccaggaggagtcatgcagccatgagtgcaaccagcggttggtcgtactgtttggggtgggaaagcagcgagatgatgcccgccatgccatcaagaaaatcaccaaggatatcttgaaggttctgaaccgcaaagggacagcagaaactgaccagcttgctcctattgtgcctctgaatcctggagacctgacattcttaggtggggaggatgggcagaagcggcgacgcaaccggcctgaagccttccccactgctgaagatatctttgctaagttccagcacctttcacattatgaccaacaccaggtcacggctcaggtctcccggaatgttctggagcagatcacgagctttgcccttggcatgtcataccacttgcctctggtgcagcatgtgcagttcatcttcgacctcatggaatattcactcagcatcagtggcctcatcgactttgccattcagctgctgaatgaactgagtgtagttgaggctgagctgcttctcaaatcctcggatctggtgggcagctacactactagcctgtgcctgtgcatcgtggctgtcctgcggcactatcatgcctgcctcatcctcaaccaggaccagatggcacaggtctttgaggggctgtgtggcgtcgtgaagcatgggatgaaccggtccgatggctcctctgcagagcgctgtatccttgcttatctctatgatctgtacacctcctgtagccatttaaagaacaaatttggggagctcttcagcgacttttgctcaaaggtgaagaacaccatctactgcaacgtggagccatcggaatcaaatatgcgctgggcacctgagttcatgatcgacactctagagaaccctgcagctcacaccttcacctacacggggctaggcaagagtcttagtgagaaccctgctaaccgctacagctttgtctgcaatgcccttatgcacgtctgtgtggggcaccatgatcccgatagggtgaatgacatcgcaatcctgtgtgcagagctgaccggctattgcaagtcactgagtgcagaatggctaggagtgcttaaggccttgtgctgctcctctaacaatggcacttgtggtttcaacgatctcctctgcaatgttgatgtcagtgacctatcttttcatgactcgctggctacttttgttgccatcctcatcgctcggcagtgtttgctcctggaagatctgattcgctgtgctgccatcccttcactccttaatgctgcttgtagtgaacaggactctgagccaggggcccggcttacctgccgcatcctccttcaccttttcaagacaccgcagctcaatccttgccagtctgatggaaacaagcctacagtaggaatccgctcctcctgcgaccgccacctgctggctgcctcccagaaccgcatcgtggatggagccgtgtttgctgttctcaaggctgtgtttgtacttggggatgcggaactgaaaggttcaggcttcactgtgacaggaggaacagaagaacttccagaggaggagggaggaggtggcagtggtggtcggaggcagggtggccgcaacatctctgtggagacagccagtctggatgtctatgccaagtacgtgctgcgcagcatctgccaacaggaatgggtaggagaacgttgccttaagtctctgtgtgaggacagcaatgacctgcaagacccagtgttgagtagtgcccaggcgcagcgcctcatgcagctcatttgctatccacatcgactgctggacaatgaggatggggaaaacccccagcggcagcgcataaagcgcattctccagaacttggaccagtggaccatgcgccagtcttccttggagctgcagctcatgatcaagcagacccctaacaatgagatgaactccctcttggagaacatcgccaaggccacaatcgaggttttccaacagtcagcagagacagggtcatcttctggaagtactgcaagcaacatgcccagcagcagcaagaccaagcctgtgctcagctctctagagcgctctggtgtatggctggtggcccccctcattgctaaactgcccacctcagtccagggacatgtgttaaaggctgctggggaagaattggagaagggtcagcacctgggttcctcttcacgcaaagaacgtgatcgacaaaagcagaagagcatgtccctattgagccagcagcccttcttatcgctggtgctaacatgtctgaaagggcaggatgaacaacgcgagggactccttacctccctctacagccaggtgcaccagattgtgaataattggcgagatgaccagtacttagatgattgcaaaccaaagcagcttatgcatgaggcactcaaactgcggctcaacctggtggggggcatgtttgacacggtgcagcgcagcacccagcagaccacggagtgggccatgctcctcctggagatcatcatcagcggcactgtcgacatgcagtccaacaatgagctcttcactactgtgttggacatgctgagcgtgctcatcaatgggacattggctgcagacatgtctagcatctcgcaaggtagcatggaggaaaacaagcgtgcatacatgaacctggcgaagaagttgcagaaggagttgggggagcgccagtcagacagtctggaaaaggttcgccagctgctgccactgcccaagcagacccgagatgtcatcacgtgtgagccacagggctcccttatcgataccaagggcaacaagattgctggcttcgattccatcttcaagaaggagggtctacaggtttccaccaaacagaagatctcgccctgggatctttttgaggggttgaagccgtcagcaccactctcttggggctggtttggaacagtccgagtggaccggcgagtggctcgaggagaggagcagcagcggttgctgctctaccacacacacctgaggccccggccccgcgcctattacctggagccactgccactgcccccagaagatgaggagccgcctgctcctaccctgctagagcctgagaaaaaggctccagagccccccaaaactgacaaaccgggggctgctccacccagtactgaggaacgcaagaagaagtccaccaagggcaagaaacgcagccagccagctaccaagacagaggactatggaatgggcccgggtcggagcggcccttatggtgtgacagtgcctccggacctcctgcaccacccaaaccctggttctataacacaccttaactacaggcaaggctccataggcctgtacacccagaaccagccactacctgcaggtggccctcgtgtggacccataccgtcctgtgcgcttaccaatgcagaagctgcccacccgaccaacttaccctggagtgctgcccacaaccatgactggcgtcatgggtttagaaccctcctcttataagacctctgtgtaccggcagcagcaacctgcggtgccccaaggacagcgccttcgccaacagctccagcagagtcagggcatgttgggacagtcatctgtccatcagatgactcccagctcttcctacggtttgcagacttcccagggctatactccttatgtttctcatgtgggattgcagcaacacacaggccctgcaggtaccatggtgccccccagctactccagccagccttaccagagcacccacccttctaccaatcctactcttgtagatcctacccgccacctgcaacagcggcccagtggctatgtgcaccagcaggcccccacctatggacatggactgacctccactcaaaggttttcacaccagacactgcagcagacacccatgataagtaccatgactccaatgagtgcccagggcgtccaggcaggcgtccgttcaacagccatcctacctgagcagcagcagcagcagcaacagcagcaacagcaacagcagcagcagcagcaacagcaacagcagcagcagcagcagcagtaccacatccggcagcagcagcagcagcagatcctgcggcagcagcagcaacagcaacagcagcagcagcagcagcagcaacagcaacagcagcagcagcaacagcaacaacagcaacaccagcagcaacagcagcaacaggcggctcctccccaaccccagccccagtcccagccccagttccagcgccaggggcttcagcagacccagcagcagcaacagacagcagctttggtccggcaacttcaacaacagctctctaatacccagccacagcccagtaccaacatatttggacgctactgagccacctggaggaactgcttgtgcactggatgtggccccaccctttcctcttaattcccaatcccattcctgggctagcaccagtagtggttggggccctcccctcaggctccatttttaataagtttttagtatttttgttaatgtgaggcattgagctgttgggttttgtatattatttatatagagaccccagagctgttgcacccaatacacagagcttctttgcaaagggaaaaaaaaaaaaaaaaa

SEQ ID NO: 2 - protein, 2177 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

SEQ ID NO: 3 - DNA, 20 bp, Artificial Sequence
  Forward 1 - primer used to amplify the desired PCR fragment of the genomic template DNA
ccccttcccctaaggaaaaa

SEQ ID NO: 4 - DNA, 20 bp, Artificial Sequence
  Reverse 1 -  primer used to amplify the desired PCR fragment of the genomic template DNA and for the amplification of the target cDNA-fragment
atgctcatccccagagacag

SEQ ID NO: 5 - DNA, 20 bp, Artificial Sequence
  Forward 2 - primer used for amplification of the target cDNA-fragment
cttcgggatcttgagctacg

SEQ ID NO: 6 - DNA, 22 bp, Artificial Sequence
  Oligonucleotide used to amplify the desired PCR fragment of the canine template DNA as well as the canine target cDNA-fragment
gatgaactgacagccttgaatg

SEQ ID NO: 7 - DNA, 21 bp, Artificial Sequence
  Oligonucleotide used to amplify the desired PCR fragment of the canine template DNA as well as the canine target cDNA-fragment
cttggcaggattgaagttgac